(19) United States
(12) Patent Application Publication
(10) Pub. No.: US 2004/0030330 A1
(43) Pub. Date: Feb. 12, 2004

(54) Electrosurgery systems

(76) Inventors: James L. Brassell, Boulder, CO (US); Steven W. Ek, Bolton, MA (US); Keith D. Foskitt, Westford, MA (US); Alan P. Gannon, Amesbury, MA (US); Warren P. Heim, Boulder, CO (US); Richard S. Lansil, Woburn, MA (US); James E. McCay, Fairfield, CT (US); Gary R. McCarthy, East Bridgewater, MA (US); Michael D. Olichney, Lyons, CO (US); Alan L. Oslan, Bedford, MA (US); Paul Sabin, Needham, MA (US); Gary Tallent, Gloucester, MA (US); Javier Verdura, Marietta, GA (US); Guy Wilkins, Cincinnati, OH (US); David L. Lockwood, Gouverneur, NY (US)

Correspondence Address: Joel R Petrow, Chief Patent Counsel, Smith & Nephew Inc, 1450 Brooks Road, Memphis, TN, 38116, US

(21) Appl. No.: 10/381,746

(22) PCT Filed: Apr. 18, 2002
(86) PCT No.: PCT/US02/12065

(51) Int. Cl. A61B 18/14

(52) U.S. Cl. 606/41; 606/48; 606/50

(57) ABSTRACT

A surgical device includes a return electrode, an active electrode, and an insulating region adjacent to the active electrode. A rasping surface is provided by either the active electrode or the insulating region. Another surgical device includes an adapter configured to couple to a generator and to convert monopolar output from the generator into bipolar output. A method of applying electricity to tissue includes bringing a surgical device into close proximity with tissue and applying electricity to the tissue using the surgical device.

### BACKGROUND

The invention relates to electrosurgery systems and, more particularly, to the use of electrosurgery in arthroscopy.

In electrosurgery, electrical energy, such as, for example, high frequency and radio frequency electrical energy, is used to modify the structure of tissue. For example, an electrical current can be directed from a first electrode (an active electrode) to a second electrode (a return electrode), and the path of the current can be used to cut, coagulate, and ablate tissue.

Electrosurgery is performed using monopolar instruments and bipolar instruments.

With a monopolar instrument, electrical current is directed from an active electrode positioned at the tissue to be treated, through the patient's body to a return electrode generally in the form of a ground pad attached to the patient. With a bipolar instrument, both the active electrode and the return electrode are positioned at the tissue to be treated, and electrical current flows from the active electrode to the return electrode over a short distance.

### SUMMARY

Aspects of the invention relate to surgical systems and instruments, such as, for example, those that are used in the field of electrosurgery. For example, the surgical systems and instruments are used for arthroscopic surgical procedures, such as resection, ablation, excision of soft tissue, hemostasis of blood vessels and coagulation of soft tissue in patients requiring arthroscopic surgery of the knee, shoulder, ankle, elbow, wrist, or hip. In some embodiments, the invention features single-use instruments used with a conductive irrigating solution, such as saline and Ringer's lactate.

According to one aspect, a surgical device includes an insulating region having a surface with a formation for providing a mechanical rasping action against tissue. The surgical device includes an active electrode, and the insulating region is adjacent the active electrode.

Embodiments of this aspect may include one or more of the following features. The formation includes a groove. The formation includes a ridge. The ridge has a flat top-surface. The ridge has a curved top-surface. The formation includes at least one of a scallop, an edge, and a point. The insulating region substantially encircles a periphery of the active electrode. The insulating region includes an electrically non-conductive, refractory material. The active electrode includes a location that provides for concentration of current density. The active electrode includes a geometry having at least one location particularly adapted to provide light off. The location includes a raised portion.

The surgical device includes a hand wand and a shaft rotatably coupled to the hand wand, and the shaft includes the active electrode and the insulating region. The shaft is continuously rotatable, such that the active electrode is continuously rotatable. The shaft defines an aspiration lumen. The surgical device includes a tube coupled to the shaft, and a suction control coupled to the tube. The tube defines a lumen in communication with the aspiration lumen and the suction control is for controlling suction through the aspiration lumen. The control includes a valve. The surgical device includes a rotation control coupled to the shaft for rotating the shaft. The rotation control includes a hand-actuated knob. The surgical device includes a power control coupled to the hand wand for controlling power applied to the active electrode. The power control includes a push button.

An electrical characteristic of the surgical device is substantially uniform around a periphery of the active electrode when the electrical characteristic is measured in a plane. The plane is perpendicular to an engagement angle between the active electrode and a tissue surface, and the plane goes through part of the active electrode. The electrical characteristic includes electric field strength. The engagement angle includes an angle providing substantially maximum tissue contact between the active electrode and a flat tissue surface. The active electrode includes a surface configured to contact tissue at an angle that is not parallel to a longitudinal axis of the surgical device.

An electrical characteristic of the surgical device measured at any point in a given plane that is at least 3/100 of an inch outside of an envelope of an active electrode drops off to no more than 60% of a maximum value for the electrical characteristic in the given plane. The active electrode defines the envelope in the given plane. The given plane goes through the active electrode. The electrical characteristic includes electric field strength. The given plane is perpendicular to an engagement angle between the active electrode and a tissue surface. The engagement angle provides substantially maximum tissue contact between the active electrode and a flat tissue surface. The active electrode includes a surface configured to contact tissue at an angle that is not parallel to a longitudinal axis of the surgical device.

An electrical characteristic of the surgical device measured at any point in a plane corresponding to a tissue depth of at least 3/100 of an inch drops off to no more than 60% of a maximum value in the plane. The active electrode contacts a tissue surface. The plane goes through the active electrode and the tissue surface. The electrical characteristic includes electric field strength. The electric field strength drops off to no more than half the maximum value at any point in the plane corresponding to a tissue depth of at least 15/1000 of an inch. The plane is parallel to an engagement angle between the active electrode and the tissue surface.

The active electrode defines an envelope in a given plane, the given plane goes through the active electrode and an electrical characteristic of the surgical device achieves a maximum for the given plane outside of the envelope. The surgical device includes a return electrode. The surgical device includes an adapter electrically coupled to the active electrode and the return electrode. The adapter is configured (i) to couple to a generator, (ii) to convert monopolar output from the generator into bipolar output, and (iii) to provide the bipolar output to the active electrode. The adapter is further configured to convert substantially constant power output from the generator into substantially constant voltage output.

According to another aspect, a method includes rasping tissue mechanically using a formation on a surface of an insulating region. The method includes applying electrical energy to tissue using an active electrode of a surgical device, and the insulating region is adjacent the active electrode.

Embodiments of this aspect may include one or more of the following features. Rasping tissue includes using a ridge as the formation. Applying electrical energy includes using a location on the active electrode, the location being particularly adapted to provide light off. Rasping tissue includes providing a user of the surgical device tactile feedback from tissue. The method includes penetrating a joint in a body with the active electrode and the formation of the surgical device. The method includes ablating tissue with the applied electrical energy. The method includes coagulating tissue with the applied electrical energy.

According to another aspect, a surgical device includes an insulating region having a surface adapted for providing a mechanical rasping action against tissue. The surgical device includes an active electrode, and the insulating region is adjacent the active electrode.

According to another aspect, a surgical device includes an insulating region having a roughened surface for providing a mechanical rasping action against tissue. The surgical device includes an active electrode, and the insulating region is adjacent the active electrode.

According to another aspect, a surgical device includes an active electrode, and an electrical characteristic of the surgical device achieves a maximum for a given plane outside of an envelope defined by the active electrode in the given plane. The given plane goes through the active electrode.

Embodiments of this aspect may include one or more of the following features. The electrical characteristic is substantially uniform around a periphery of the active electrode when the electrical characteristic is measured in the given plane. The given plane is perpendicular to an engagement angle between the active electrode and a tissue surface. The electrical characteristic measured at any point in the given plane that is at least 3/100 of an inch outside of the envelope drops off to no more than 60% of a maximum value for the electrical characteristic in the given plane.

According to another aspect, a surgical device includes a hand wand and a shaft rotatably coupled to the hand wand and continuously rotatable with respect to the hand wand. The shaft is adapted to be inserted into a joint in a body.

Embodiments of this aspect may include one or more of the following features. The surgical device includes a rotation control coupled to the shaft for rotating the shaft. The shaft defines an aspiration lumen and the surgical device includes a tube coupled to the shaft and a suction control coupled to the tube. The tube defines a lumen in communication with the aspiration lumen, and the suction control is for controlling suction through the aspiration lumen. The surgical device includes an active electrode coupled to the shaft. The surgical device includes a power control coupled to the hand wand for controlling power applied to the active electrode. The rotation control includes a knob. The suction control includes a valve. The power control includes a push button.

According to another aspect, a method includes inserting a shaft of a surgical device into a joint in a body, the shaft being rotatably coupled to a grip, and rotating the shaft through more than 360 degrees in one direction without rotating the grip.

Embodiments of this aspect may include one or more of the following features. The method includes aspirating fluid through a lumen defined by the shaft, and controlling the aspirating using an aspiration control coupled to the grip. The method includes applying electrical power to an active electrode coupled to the shaft, and controlling the power using a power control coupled to the grip.

According to another aspect, a system includes an adapter that includes first circuitry to convert monopolar output from a generator into bipolar output for an active electrode. The adapter is configured to be electrically coupled to the active electrode and to the generator.

Embodiments of this aspect may include one or more of the following features. The first circuitry is adapted to convert substantially constant power output from the generator into substantially constant voltage output. The adapter is configured to be electrically coupled to a return electrode, and the adapter includes second circuitry to receive bipolar return from the return electrode. The first circuitry and the second circuitry overlap such that each of the first circuitry and the second circuitry include a specific circuit element. The system includes the active electrode and the return electrode, the active electrode and the return electrode both being electrically coupled to the adapter.

According to other aspects, the invention relates to methods and apparatus for rasping tissue while applying electrical energy to the tissue.

Advantages of the invention may include (i) providing a surgeon tactile feedback as well as the ability to move or disrupt tissue by providing a rasping formation on a surgical tip, (ii) allowing access to tissue at different sites within a body by providing different surgical tips and a rotatable surgical tip, (iii) allowing a surgeon to effectively operate on tissue by providing relatively uniform electrical characteristics around the entire perimeter of an electrode, and by providing a high electric field strength outside of and/or above the envelope of an electrode, (iv) reducing the risk of burning tissue below the surface tissue that is of interest by providing an electric field strength or other electrical characteristic that falls off quickly within tissue, (v) minimizing the possibility of runaway current during electrosurgery by providing an adapter that converts constant power output from a generator to constant voltage output for an electrosurgical probe, (vi) simplifying endoscopic operations by providing suction to remove debris and bubbles to maintain a clear view of the target tissue, (vii) simplifying endoscopic operations by providing a surgical instrument with a hand grip that includes controls for power, suction, and/or rotation, and (viii) reducing patient burn and other disadvantages of monopolar devices by providing a bipolar surgical device.

The details of one or more embodiments are set forth in the accompanying drawings and the description below. Other features, objects, and advantages of the invention will be apparent from the description and the drawings, and from the claims.

### DESCRIPTION OF DRAWINGS

FIG. 1 is a perspective view of an embodiment of a surgical system including a generator, an adapter module and a probe;

FIG. 2 is a perspective view of the generator of FIG. 1 with a front, exploded view of the adapter module;

FIG. 3 is a back exploded view of the adapter module of FIG. 2;

FIG. 3A shows a perspective view of the back of another adapter module;

FIG. 3B shows a perspective view of the front of the adapter module of FIG. 3A;

FIG. 3C shows a cross-sectional view of the adapter module of FIG. 3B, taken along line 3C-3C;

FIG. 3D is a front, exploded view of the adapter module of FIG. 3A;

FIG. 3E is a back, exploded view of the adapter module of FIG. 3A;

FIG. 4 is a front view of the adapter module of FIG. 2;

FIG. 5 is a cross-sectional view of the adapter module of FIG. 4, taken along line 55;

FIG. 6 is a partial schematic diagram of an embodiment of an adapter module and a probe with hand switches;

FIG. 7 is a partially cut-away, perspective view of the probe of FIG. 1;

FIG. 8 is a detailed, partially cut-away, perspective view of the probe of FIG. 1;

FIG. 9 is a detailed cross-sectional view of the probe of FIG. 1;

FIG. 10 illustrates the wiring of the probe of FIG. 1;

FIG. 11 is a partial schematic diagram of an embodiment of a probe without hand switches;

FIGS. 12A and 12B are perspective views of an embodiment of a valve housing;

FIG. 13 is a perspective view of an embodiment of a valve actuator;

FIG. 14 is a cross-sectional view of an embodiment of a valve;

FIGS. 15-15H are perspective views of various embodiments of a surgical tip;

FIG. 16 is a perspective view of an embodiment of a surgical tip;

FIG. 16A is an exploded perspective view of the surgical tip of FIG. 16;

FIG. 16B is a top view of the surgical tip of FIG. 16;

FIG. 16C is a cross-sectional view of the surgical tip of FIG. 16B, taken along line 16C-16C;

FIG. 16D is a cross-sectional end view of the surgical tip of FIG. 16C, taken along line 16D-16D;

FIG. 16E is a cross-sectional view of the surgical tip of FIG. 16B, taken along line 16E-16E;

FIG. 16F is a cross-sectional end view of the surgical tip of FIG. 16E, taken along line 16F-16F;

FIG. 16G is a perspective view of another embodiment of a surgical tip;

FIG. 16H is an exploded perspective view of the surgical tip of FIG. 16G;

FIG. 16I is a top view of the surgical tip of FIG. 16G;

FIG. 16J is a cross-sectional view of the surgical tip of FIG. 16I, taken along line 16J-16J;

FIG. 16K is an end view of the surgical tip of FIG. 16G;

FIG. 17 is a perspective view of another embodiment of a surgical tip;

FIG. 17A is an exploded perspective view of the surgical tip of FIG. 17;

FIG. 17B is a top view of the surgical tip of FIG. 17;

FIG. 17C is a cross-sectional view of the surgical tip of FIG. 17B, taken along line 17C-17C;

FIG. 17D is an end view of the surgical tip of FIG. 17;

FIG. 18 is a perspective view of another embodiment of a surgical tip;

FIG. 18A is an exploded perspective view of the surgical tip of FIG. 18;

FIG. 18B is a top view of the surgical tip of FIG. 18;

FIG. 18C is a cross-sectional view of the surgical tip of FIG. 18B, taken along line 18C-18C;

FIG. 18D is a cross-sectional view of the surgical tip of FIG. 18C, taken along line 18D-18D;

FIG. 18E is an end view of the surgical tip of FIG. 18;

FIG. 19 is a perspective view of another embodiment of a surgical tip;

FIG. 19A is an exploded perspective view of the surgical tip of FIG. 19;

FIG. 19B is a top view of the surgical tip of FIG. 19;

FIG. 19C is a cross-sectional view of the surgical tip of FIG. 19B, taken along line 19C-19C;

FIG. 19D is a cross-sectional view of the surgical tip of FIG. 19C, taken along line 19D-19D;

FIG. 19E is an end view of the surgical tip of FIG. 19;

FIG. 20 is a perspective view of another embodiment of a surgical tip;

FIG. 20A is an exploded perspective view of the surgical tip of FIG. 20;

FIG. 20B is a top view of the surgical tip of FIG. 20;

FIG. 20C is a cross-sectional view of the surgical tip of FIG. 20B, taken along line 20C-20C;

FIG. 20D is an end view of the surgical tip of FIG. 20;

FIGS. 21A-C are perspective, top, and side views, respectively, of an embodiment of an electrode:

FIG. 22 is a perspective view of another embodiment of a surgical tip;

FIG. 22A is an exploded perspective view of the surgical tip of FIG. 22;

FIG. 22B is a top view of the surgical tip of FIG. 22;

FIG. 22C is a cross-sectional view of the surgical tip of FIG. 22B, taken along line 22C-22C;

FIG. 22D is an end view of the surgical tip of FIG. 22;

FIG. 23 is a perspective view of another embodiment of a surgical tip;

FIG. 23A is an exploded perspective view of the surgical tip of FIG. 23;

FIG. 23B is a top view of the surgical tip of FIG. 23;

FIG. 23C is a cross-sectional view of the surgical tip of FIG. 23B, taken along line 23C-23C;

FIG. 23D is an end view of the surgical tip of FIG. 23;

FIGS. 24A-C are perspective, top, and side views, respectively, of another embodiment of an electrode;

FIG. 25 is a longitudinal cross-sectional view of another embodiment of a surgical tip, taken along the same line as FIG. 16C;

FIG. 25A is a longitudinal cross-sectional view of the surgical tip of FIG. 25, taken along the same line as FIG. 16E;

FIG. 25B is a radial cross-sectional view of the surgical tip of FIG. 25, taken along line 25B-25B;

FIG. 25C is a radial cross-sectional view of the surgical tip of FIG. 25A, taken along line 25C-25C;

FIG. 26 is a perspective view of another assembled surgical tip;

FIG. 26A is an exploded perspective view of the surgical tip of FIG. 26;

FIG. 26B is a top view of the surgical tip of FIG. 26;

FIG. 26C is a longitudinal cross-sectional view of the surgical tip of FIG. 26B, taken along line 26C-26C;

FIG. 26D is a longitudinal cross-sectional view of the surgical tip of FIG. 26C, taken along line 26D-26D;

FIG. 26E is a distal end view of the surgical tip of FIG. 26.

FIG. 27 is a perspective view of another assembled surgical tip;

FIG. 27A is an exploded perspective view of the surgical tip of FIG. 27;

FIG. 27B is a top view of the surgical tip of FIG. 27;

FIG. 27C is a longitudinal cross-sectional view of the surgical tip of FIG. 27B, taken along line 27C-27C;

FIG. 27D is an enlarged portion of FIG. 27C;

FIG. 27E is a distal end view of the surgical tip of FIG. 27;

FIG. 28 is perspective view of a housing of another surgical tip;

FIG. 28A is a perspective view of an electrode for use with the housing of FIG. 28;

FIG. 29 is a perspective view of another assembled surgical tip;

FIG. 29A is an exploded perspective view of the surgical tip of FIG. 29;

FIG. 29B is a top view of the surgical tip of FIG. 29;

FIG. 29C is a longitudinal cross-sectional view of the surgical tip of FIG. 29B, taken along line 29C-29C;

FIG. 29D is an enlarged portion of FIG. 29C;

FIG. 29E is a distal end view of the surgical tip of FIG. 29;

FIG. 30 includes a graph of isometric lines of electric potential for the surgical tip of FIGS. 25-25F;

FIG. 31 includes a graph of electric field vectors for the surgical tip in the graph in FIG. 30;

FIG. 32 includes a graph of electric field vectors for the surgical tip of FIGS. 18-18E;

FIG. 33 includes a graph of isometric lines of electric potential for a portion of the surgical tip of FIGS. 27-27E; and

FIG. 34 includes a graph of electric field vectors for the portion of the surgical tip in the graph in FIG. 33.

All dimensions shown and materials listed in the figures are illustrative and not intended to be limiting. Distance dimensions are in inches unless otherwise noted.

### DETAILED DESCRIPTION

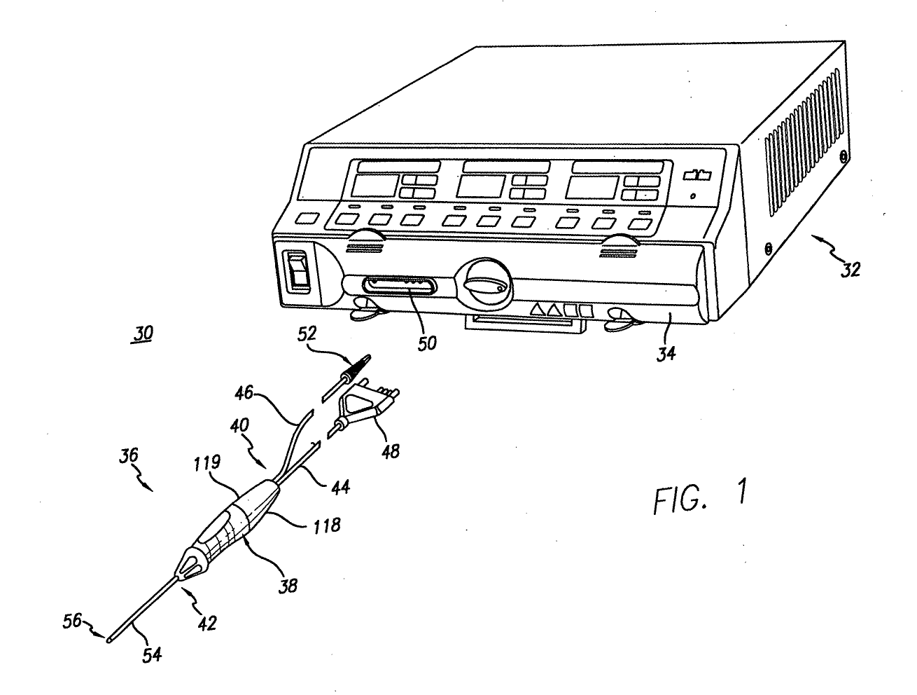

Referring to FIG. 1, a surgical system 30 includes a generator 32, an adapter module 34 connectable to generator 32, and a radio frequency bipolar probe 36 connectable to adapter module 34. Probe 36 includes a hand wand 38 having a proximal end 40 and a distal end 42. Wand 38 has a cable 44 and a suction tube 46 extending from its proximal end 40. Cable 44 terminates with a male connector 48, and suction tube 46 terminates with a suction barb connector 52. Male connector 48 is configured to mate with a female receptacle 50 defined by module 34. At its distal end 42, wand 38 has a rotation tube 54, e.g., made of stainless steel, extending therefrom and terminating at a surgical tip 56, having, for example, an active electrode. The length of rotation tube 54 is electrically insulated, e.g., with a heat shrink polymer, except a portion of the rotation tube near tip 56 is uninsulated to serve as a return electrode.

Generally, generator 32 provides constant electric power to adapter module 34, which converts the power to a form useable by probe 36, e.g., approximately constant voltage. The converted power is sent to surgical tip 56 via cable 44, wand 38, and rotation tube 54. By manipulating probe 36 at a tissue site and selectively applying power, a surgeon can use surgical system 30 for electrosurgery.

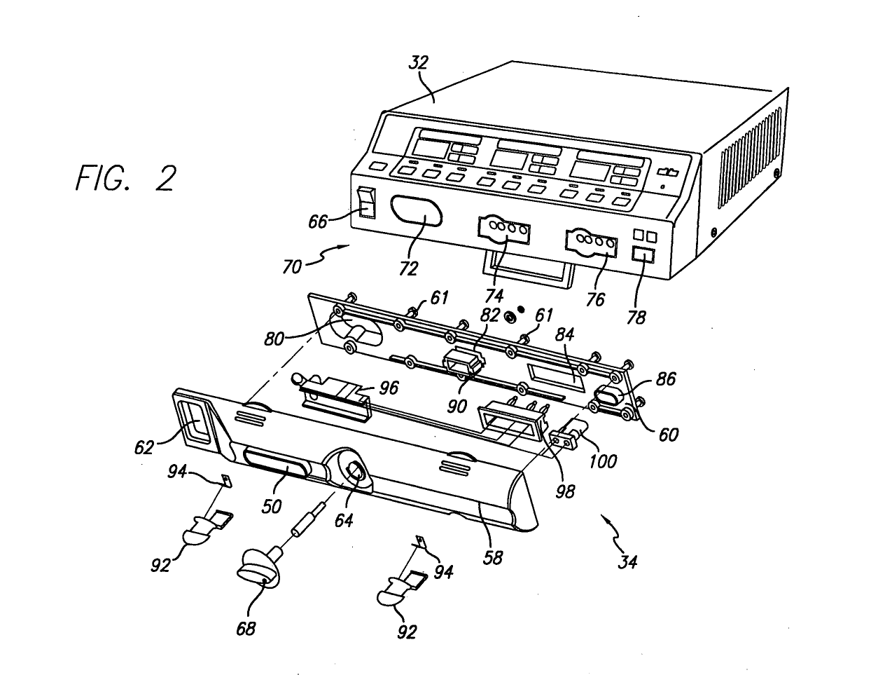

Referring to FIG. 2, generator 32 has a front portion 70 that includes a power switch 66, a bipolar current output 72, a first monopolar current output 74, a second monopolar current output 76, and a return current input 78. Generator 32 can be a commercially available generator, such as a Force FX™/Force FX™-C generator, available from Valleylab Inc., Boulder, Colo.

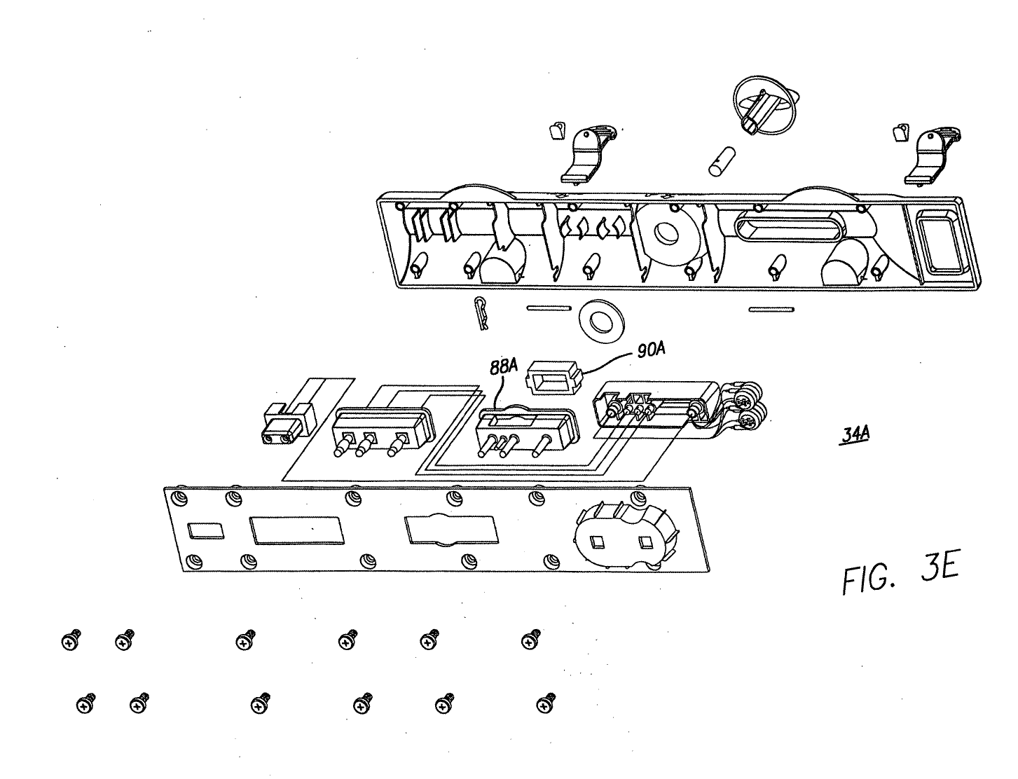

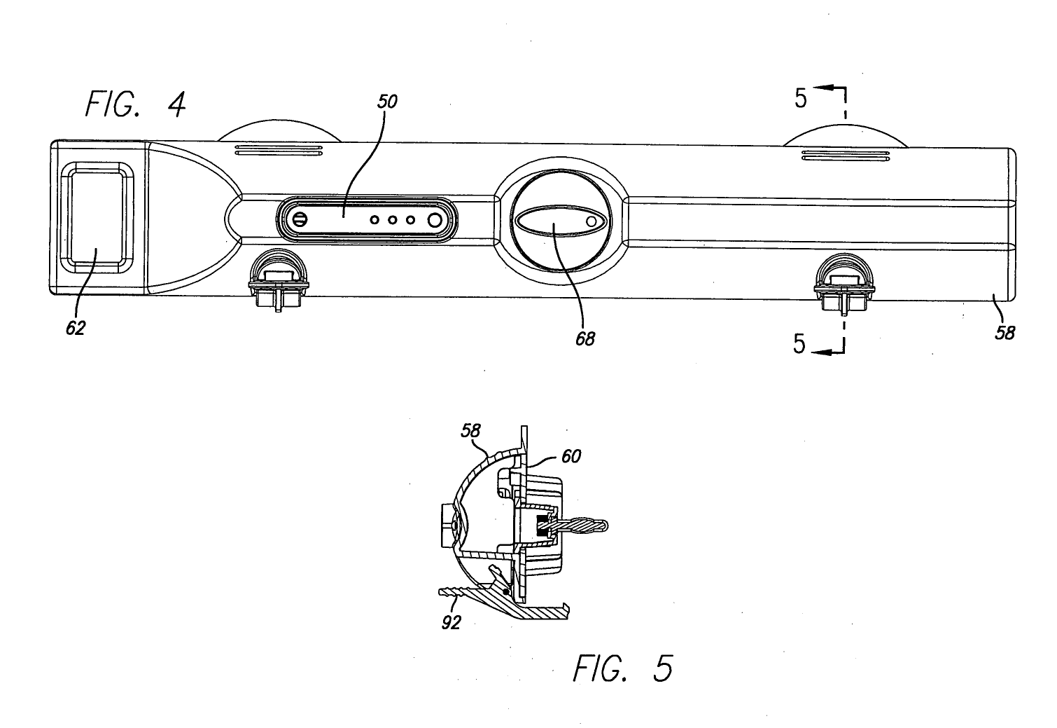

Referring to FIGS. 2-5, adapter module 34 has a unibody design that simultaneously establishes all appropriate connections to generator 32 and blocks unwanted connections. Adapter module 34 can be a commercially available adapter, such as a Dyonics® Control RF Generator Adaptor, available from Smith & Nephew, Andover, Mass. Adapter module 34 is configured to attach to front portion 70 of generator 32, and to convert the constant power output from the generator to a constant voltage output to probe 36, thereby minimizing the possibility of runaway current during use. Module 34 includes a front plate 58 and a back plate 60 that, when connected together with screws 61, form a housing for the module. Back plate 60 includes a covered recess 80, a central opening 82, a current output opening 84, and a current input opening 86. Recess 80 is configured to engage bipolar current output 72, thereby blocking the bipolar current output and preventing probe 36 from being used with an inappropriate power output, e.g., bipolar current. Around central opening 82, back plate 60 is connected to a housing 88. Housing 88 mates with first monopolar current output 74 of generator 32. Similar to recess 80, housing 88 is configured to block first monopolar current output 74 and to prevent probe 36 from being used with an inappropriate power output. Housing 88 and recess 80 can also serve as a guiding mechanism for attaching module 34 to generator 32. Housing 88 contains a member 90 made of a resilient and expandable material such as Santoprene rubber. As will soon be described, member 90 provides an attachment mechanism between module 34 and generator 32. Current output opening 84 and current input opening 86 are configured to overlap with second monopolar current output 76, and return current input 78, respectively.

Front plate 58 of adapter module 34 includes a power switch opening 62, female receptacle 50, and a cam lock opening 64. Power switch opening 62 provides access to power switch 66 when module 34 is attached to generator 32 (FIG. 1). As discussed above, female receptacle 50 receives male connector 48 of probe 36. Cam lock opening 64 receives a cam lock 68, which is connected to member 90 to provide an attachment mechanism between module 34 and generator 32. During use, module 34 is placed over front portion 70 of generator 32 and attached by turning cam lock 68 from an unlock position to a lock position. This action causes portions of member 90 to expand sufficiently out of housing 88, thereby providing an interference fit between member 90 and first monopolar current output 74.

To further secure module 34 to generator 32, module 34 includes two clips 92, each connected to a leaf spring 94. Leaf springs 94 connect clips 92 to front portion 70 of generator 32, and clips 92 hook to the underside of the generator (FIG. 5).

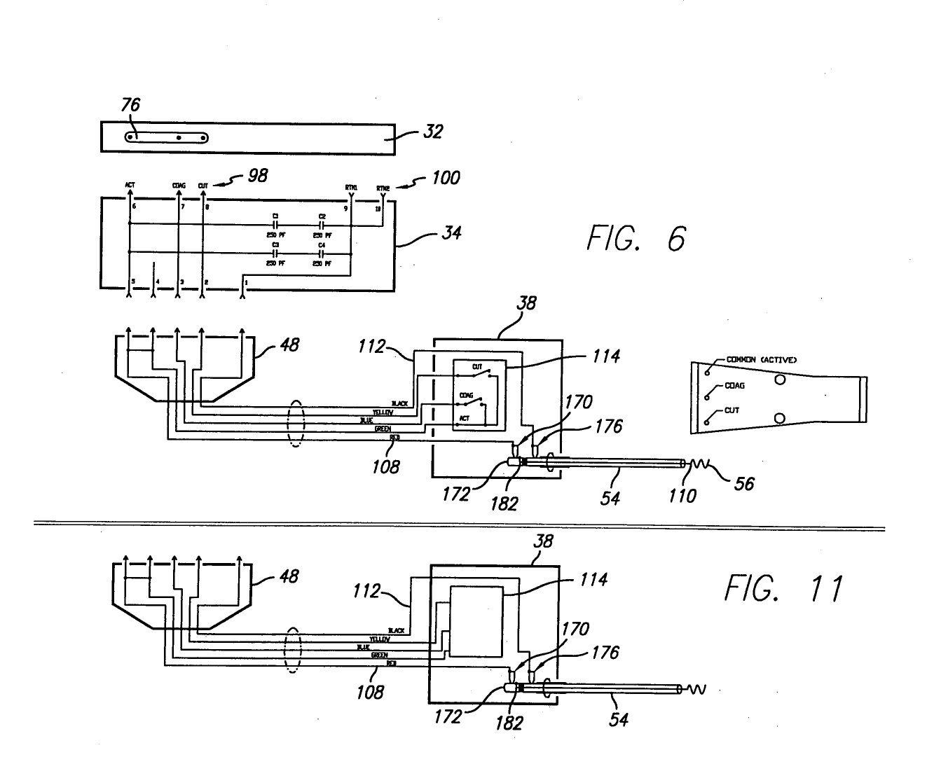

Inside its housing, module 34 includes electronic circuitry that converts constant power to constant voltage, and sends the voltage to probe 36 via male connector 48. FIG. 6 shows a schematic circuit diagram of the electronic circuitry having two sets of two capacitors. The two sets of capacitors, e.g., cera-mite high voltage capacitors (250 pF, 10,000 VDC), are placed in parallel. By placing the two sets of capacitors in parallel, the capacitors serve as voltage dividers and current limiters. Further, the capacitors provide a capacitive load that is large compared to the capacitive load near tip 56. The voltage division, current limiting, and large relative capacitive load enable the conversion from constant power to constant voltage, or substantially constant voltage.

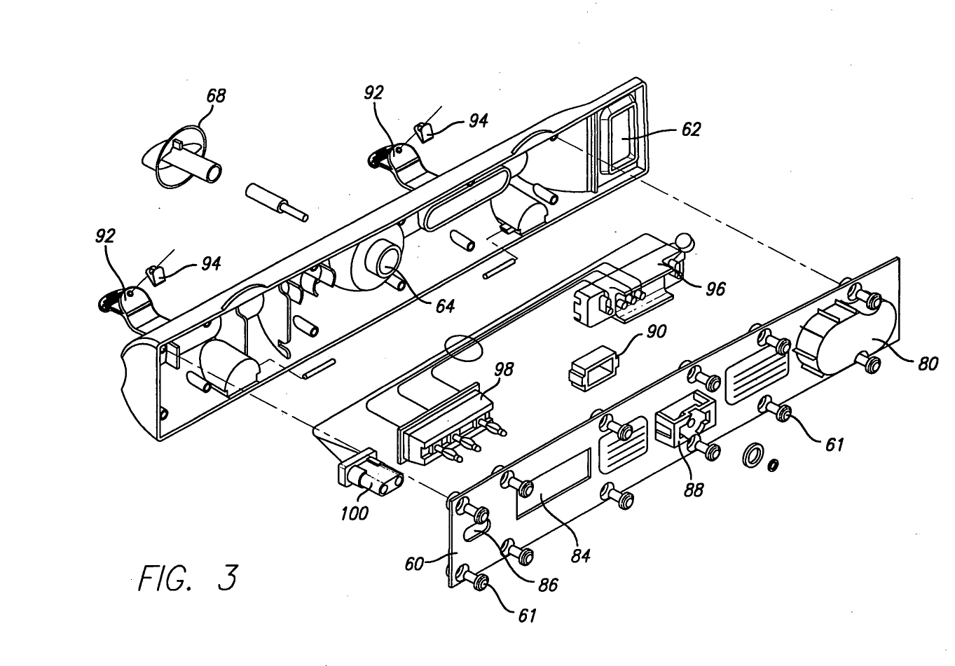

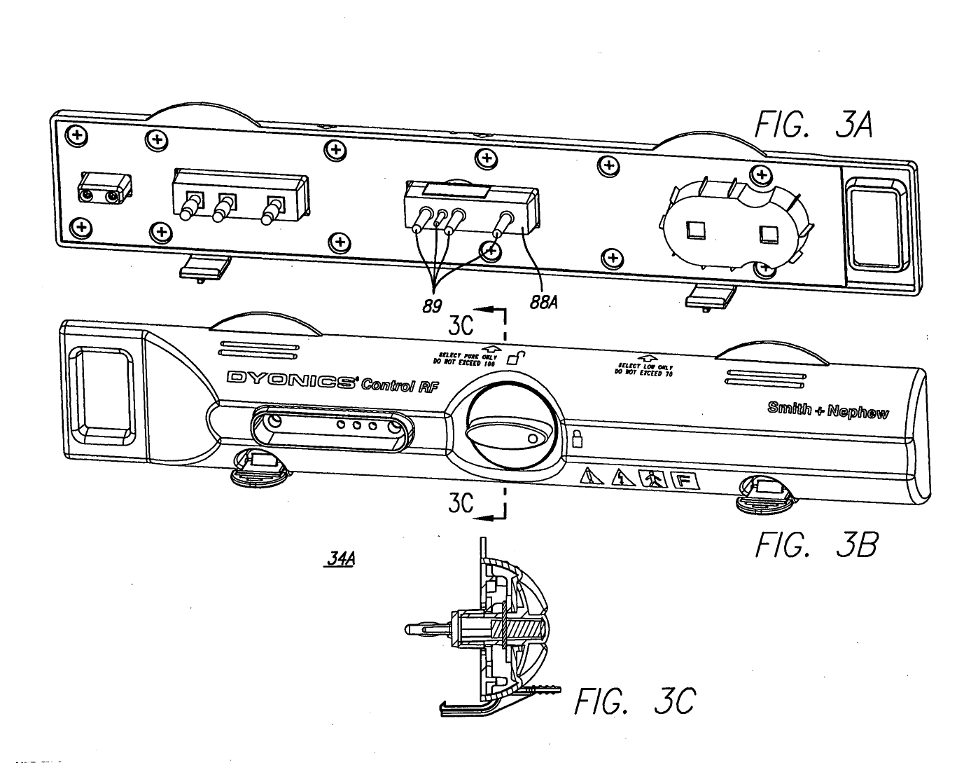

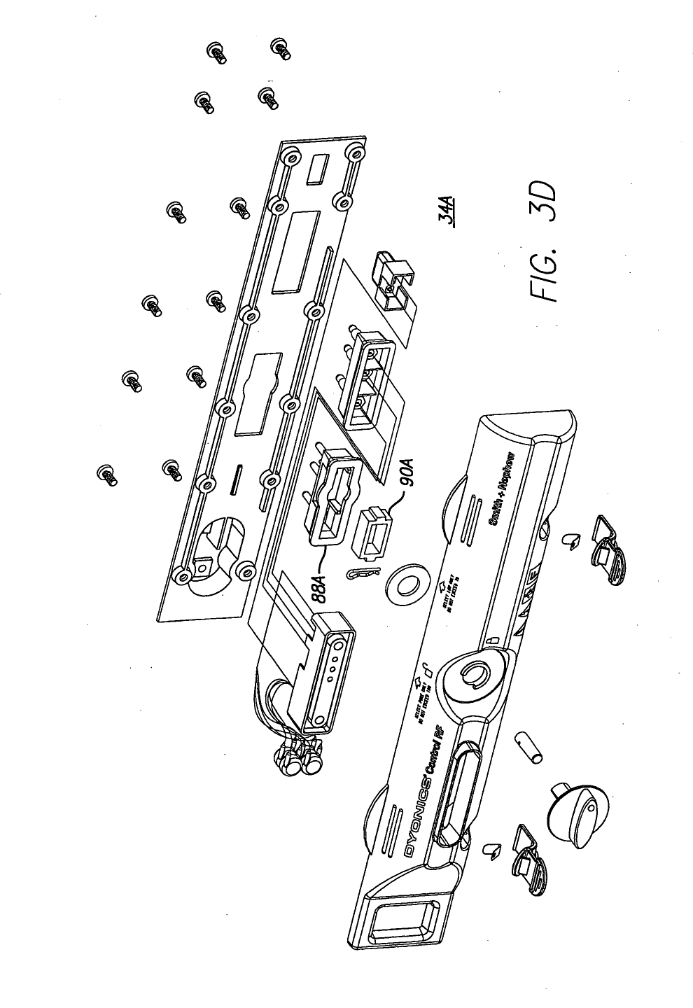

Referring again to FIGS. 2 and 3, the electronic circuitry includes a wiring harness 96 that connects to the interior side of female receptacle 50, a three-pin male connector 98 whose pins connect to second monopolar current output 76 through current output opening 84, and a two-pin male connector 100 whose pins connect to return current input 78 through current input opening 86.

Referring to FIGS. 3A-3E another adapter module 34A includes a housing 88A used in place of housing 88 (FIG. 3) to mate with first monopolar current output 74 of generator 32 (FIG. 2). Housing 88A is coupled to member 90A which may be substantially similar to member 90 (FIGS. 2-3). Housing 88A includes four projections 89 (FIG. 3A) that mate with corresponding receiving holes (see FIG. 2) in first monopolar current output 74.

Projections 89 plug into first monopolar current output 74, and at least one of projections 89, for example, an end projection, activates or selects a particular mode in generator 32. Projections 89 need not be electrical contacts, but can activate the particular mode by mechanical or other means. In one implementation, the short projection, of projections 89, activates a micro-switch in generator 32 to select the mode. Generator 32 is, for example, a Valleylab Force FX™, and the particular mode is, e.g., a reduced power mode that limits the output power for cutting to 100 Watts and for coagulating to 70 Watts. Housing 88A also serves as a guiding mechanism for attaching adapter module 34A to generator 32.

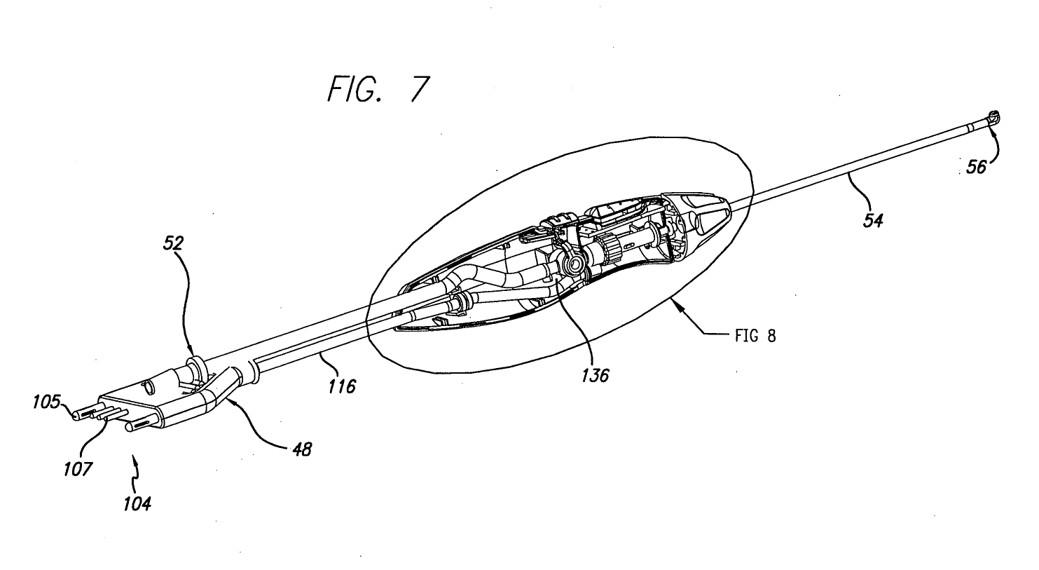

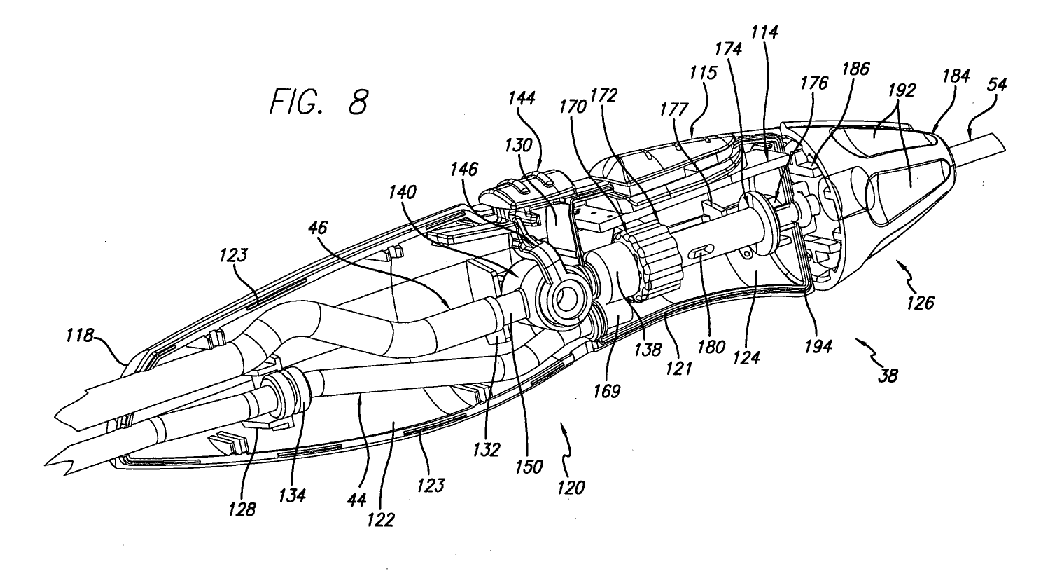

Referring to FIGS. 7-9, adapter module 34 and wand 38 are connected by cable 44 and a suction tube 46. Suction tube 46 extends from the proximal end of wand 38 to male connector 48 where the tube terminates in suction barb connector 52, which is generally not integrally formed with male connector 48 (compare FIG. 1 and FIG. 7). At its proximal end 102, cable 44 terminates in male connector 48 having five pins 104 configured to connect with sockets (not shown) in female receptacle 50 of module 34. Pins 104 include two long pins 105 at lateral ends of male connector 48, and three short pins 107 grouped offset from the center of the male connector. Pins 104 are arranged on male connector 48 such that the male connector can be inserted in female receptacle 50 in only one orientation, thereby minimizing misuse of probe 36. At its distal end 106, cable 44 terminates in wand 38. Specifically, cable 44 includes an electrically insulating outer tubing that includes an integrally-formed grommet 169 near the distal end of the cable (FIG. 8). Grommet 169 engages a rounded recess defined by a wall 130 of wand 38 to help secure cable 44 to wand 38.

Cable 44 includes five conductors that extend from pins 104 to wand 38. FIGS. 6 and 10 show schematic diagrams of the connection of conductors. Generally, an active conductor 108 is connected to an electrode 110, and a return conductor 112 is connected to rotation tube 54, an uninsulated portion of which serves as a return electrode. Three other conductors (a cut conductor, a coagulation conductor, and a second active conductor) are connected to a printed circuit board 114, which is used to control the type of power provided to electrode 110, e.g., power of different waveforms such as pulses and continuous power. Printed circuit board 114 is connected to a silicone keypad 115 provided on top of a housing 120 to provide manual control of power. Other power controls may be used, and control may be continuously variable, such as with a knob, or variable among a discrete number of options, such as with a switch. Examples of different power settings include 0-70 watts for coagulation and 0-120 watts for cutting. One implementation uses two push buttons for hand control of power, with the push buttons providing power only when pressed and held. One push button enables cut power and the other push button enables coagulation power. The same implementation optionally provides the same cut/coagulation control with a foot pedal, and controls the power setting, that is, the Watts level, at the generator.

In some embodiments, generator 32 can be equipped with a foot control, e.g., to control power. FIG. 11 shows another embodiment of a schematic diagram of the connection of the conductors. In this embodiment, a foot control is used in lieu of the circuit board to control power, so the printed circuit board is used only to terminate the conductors and is a blank board.

Referring again to FIGS. 7-9, wand 38 includes a left handle 118 and a right handle 119 (FIG. 1) that together form housing 120. Handles 118 and 119 are mirror images of each other. When left and right handles 118 and 119 are connected together, housing 120 defines a wall 130 that divides the housing into a proximal chamber 122 and a distal chamber 124. Handles 118 are connected together by ultrasonic sealing or welding. The edge perimeter of distal chamber 124 includes a continuous raised ridge 121 that acts as an energy director during ultrasonic sealing to minimize leaks, e.g., aspirated fluid, from wand 38. The edge perimeter of proximal chamber 122 includes spaced-apart ridges 123 that act as energy directors during ultrasonic sealing.

Proximal chamber 122 contains a valve 136, suction tube 46, and cable 44. Valve 136 regulates suction between suction tube 46 and surgical tip 56 (as described below). Referring to FIGS. 12A, 12B, 13 and 14, valve 136 includes a valve housing 140 and a valve actuator 146. Valve housing 140 includes a bell housing 138, a central housing 141 connected to the bell housing by a tubular bridging portion 148, and a tubular section 150 connected to the central housing. When valve 136 is assembled in an assembled probe 36, bell housing 138 is located in distal chamber 124, and central housing 139 is located in proximal chamber 122. Bell housing 138 defines a chamber 139; bridging portion 148 defines a bore 149; central housing 141 defines a chamber 143; and tubular section 150 defines a bore 151. Bores 149 are 151 are coaxial. Thus, valve housing 140 provides fluid communication between chamber 139 and bore 151 (FIG. 14). Bridging portion 148 further defines an exterior annular groove 152 that engages a rounded recess of wall 130, thereby helping to retain valve 140 in place when left and right handles 118 and 119 are connected together (FIG. 8). Tubular section 150 further defines an exterior that is configured to mate with suction tube 46. When probe 36 is fully assembled, suction tube 46 mates with tubular section 150.

Referring to FIGS. 13 and 14, valve actuator 146 is generally configured to mate with valve housing 140 to regulate suction through tube 46. In particular, valve actuator 146 includes a generally tubular portion 154 and an arm 156 connected to the tubular portion. Tubular portion 154 is configured to mate with central housing 141 and be rotatable inside the central housing. Tubular portion 154 also defines an annular groove 155 configured to receive an O-ring (not shown) to provide a tight seal between tubular portion 154 and central housing 141 when they are mated. Arm 156 is connected to a suction slide button 144 slidably positioned on top of wand 38 such that moving the slide button back and forth rotates tubular portion 154 within valve housing 140. Tubular portion 154 includes a bore 158 that extends through the tubular portion such that when valve actuator 146 mates with valve housing 140, bore 158 can align or misalign with bores 149 and 151. Thus, during use, when suction tube 46 provides a suction force to bore 151, the amount of suction force provided to bore 149 can be regulated by moving slide button 144, which controls the degree of alignment between bore 158 of actuator 146 and bores 149 and 151 of valve housing 140. For example, when slide button 144 is positioned at a most proximal position, bore 158 is completely misaligned with bores 149 and 151, and no suction force is provided to bore 149 and chamber 139. When slide button 144 is positioned at a most distal position, bore 158 is completely aligned with bores 149 and 151, and all the applied suction force provided by suction tube 46 is provided to bore 149 and chamber 139. For relatively easy movement, valve housing 140 and valve actuator 146 can be made, for example, of lubricious materials such as nylon and polycarbonate.

Referring again to FIG. 8, left and right handles 118 and 119 define support elements 128 and 132 in proximal chamber 122 that help hold cable 44 and suction tube 46, respectively, in wand 38. Support element 128 defines a rounded portion that is configured to engage a grommet 134 integrally formed with cable 44, thereby preventing cable 44 from being pulled from wand 38. Support element 132 defines a V-shaped groove (not shown) that engages tubular section 150 of valve housing 140 to help hold the housing in place, e.g., when a user slides button 144.

In distal chamber 124, wand 38 includes a conductive rear clamp 170, a conductive rear contact 172, an insulating rotation core 174, and a conductive front clamp 176. Rotation core 174 is generally a hollow tubular member. Rotation core 174 is supported, in part, by a support element 177 integrally defined by left and right handles 118 and 119. Clamps 170 and 176, shown in cross-sectional views in FIG. 10, are metallic clamps with solder tabs. Clamps 170 and 176 are attached to left handle 118. Rear clamp 170 is configured to engage with and secure rear contact 172, while still allowing the rear contact to rotate. Rear contact 172 is a metallic member having an opening at its generally flat base and a vertical corrugated wall, e.g., similar to the bundt cake pan. The opening at the base of rear contact 172 defines engaging elements, e.g., teeth, that can engage with rotation core 174, described below. The grooves and peaks defined by corrugations of rear contact 172 are spaced, in this embodiment, fifteen degrees apart. Other spacing intervals are possible. Thus, as described below, as rotation tube 54 is rotated and rear contact 172 rotates with the rotation tube, the rotation tube can be temporarily “locked”, e.g., indexed, into position every fifteen degrees via the rear contact.

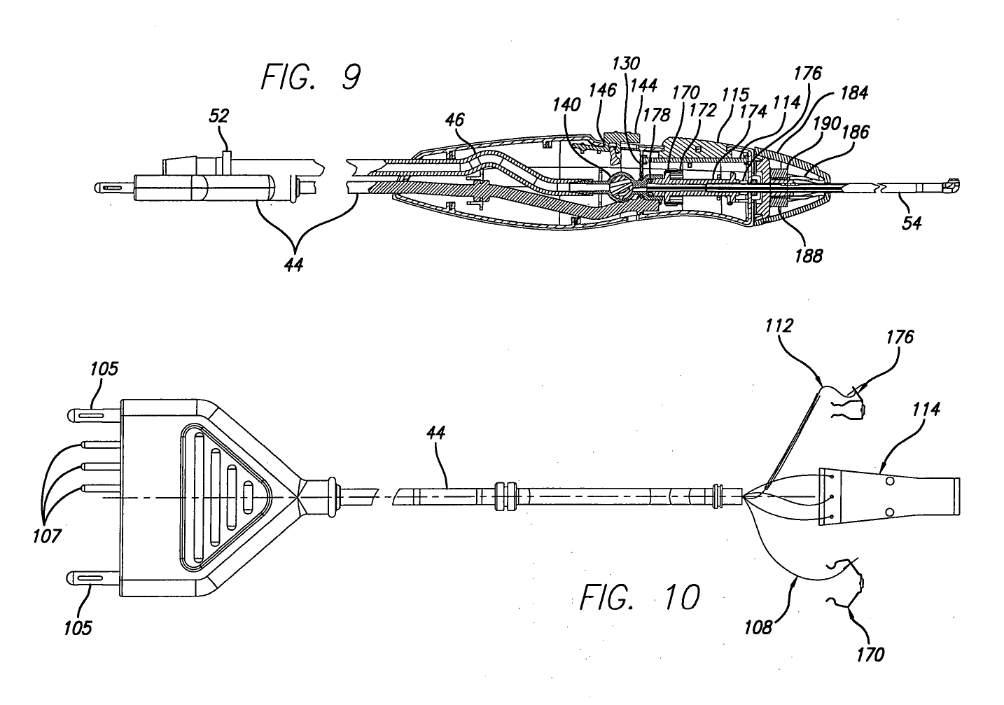

Near the proximal end of distal chamber 124, rotation core 174 is configured to mate with bell housing 138 at a proximal end and with rotation tube 54 at a distal end. Near its proximal end, rotation core 174 passes through the base opening of rear contact 172. The engaging elements defined by rear contact 172 grip rotation core 174 with a press fit such that the rear contact and the rotation core rotate together. At its proximal end, rotation core 174 mates with chamber 139 and butts against bell housing 138 (FIG. 9). Bell housing 138 includes an O-ring 178 therein to provide a tight seal between the bell housing and rotation core 174 when they engage. Bell housing 138 remains stationary, held in place in part by wall 130. At its distal end, rotation core 174 mates with the proximal end of rotation tube 54. Rotation core 174 and rotation tube 54 are securely connected, e.g., with an interference fit and/or an adhesive, such that they rotate together. Rotation core 174 defines an opening 180 that allows active electrode conductor 108 to be threaded into lumens defined by the rotation core and rotation tube 54. The active electrode conductor then makes electrical contact with an active electrode at tip 56, as described below.

Front clamp 176 is attached to left handle 118 and is configured to engage with and secure an uninsulated portion of rotation tube 54, while still allowing the rotation tube to rotate. Front-clamp 176 is connected to return electrode conductor 112, and since the front clamp and rotation tube 54 are electrically connected, the rotation tube serves as a return electrode. Front clamp 176 is generally similar to rear clamp 170 in design but smaller to engage rotation tube 54.

Referring again to FIG. 6, the electrical wiring of wand 38 is shown. Active conductor 108 extends from cable 44 and is soldered to rear clamp 170, e.g., to a solder tab. An insulated second segment of active conductor 182 is then connected, e.g., by soldering, to rear contact 172, passed through opening 180, and extended through lumens defined by rotation core 174 and rotation tube 54 to tip 56. Second segment of active conductor 182 then electrically contacts an active electrode at the distal end of rotation tube 54. By using two segments of an active conductor, rotation tube 54, rotation core 174, and rear contact 172 can be rotated freely 360 degrees, e.g., without the active conductor entangling with or wrapping around a component of wand 38. Opening 180 of rotation core 174 is sealed, e.g., with a UV-curable epoxy, to provide the lumens of rotation core 174 and rotation tube 54 with an air and liquid tight seal. Return conductor 112 extends from cable 44 and is soldered to front clamp 176, e.g., to a solder tab. Front clamp 176 clamps an uninsulated portion of rotation tube 54.

At the distal end of right and left handles 118, wand 38 includes a nose piece assembly 126 having a nose piece 184 and a nose piece mount 186. Referring to FIG. 9, nose piece mount 186, which can be made of nylon for good flex, defines a threaded portion 188 that can engage with a nut 190. Nose piece mount 186 can be securely attached to rotation tube 54 by passing the rotation tube through the nose piece mount, threading nut 190 onto portion 188, and tightening the nut. Once tightened by nut 190, nose piece mount 186 and rotation tube 54 rotate together. Rotation tube 54 also passes through nose piece 184. Nose piece 184 and nose piece mount 186 snap fit together and define interlocking elements (not shown), e.g., slots and tabs, such that, once fitted together, the nose piece and the nose piece mount rotate together with rotation tube 54. Nose piece 184 defines recesses 192 about its conical exterior to provide a good gripping surface by which to rotate rotation tube 54. By rotating nose piece 184, rotation tube 54 can be made to rotate. Further, the rotation can be continuous in a given direction because there is no wire that will bind or any other impediment to continued rotation.

Proceeding distally of probe 36, rotation tube 54 a stainless steel tube that is insulated, e.g., with a polymeric insulator such as a polyester, from about the distal end of left and right handles 118 and 119 to near the distal end of the rotation tube. The uninsulated portion of rotation tube 54 is used as a return electrode.

At its distal end, wand 38 includes surgical tip 56, e.g., a bipolar electrode, at the distal end of rotation tube 54. FIG. 15 shows multiple embodiments of surgical tips, some of which will be described in detail below. Generally, the surgical tips are configured to provide a surgeon different access to different anatomical sites. For example, tips 215, 230, 400 and 500 may be particularly useful for angled or recessed sites, such as those encountered in shoulder surgery. Tips 215, 230, and 400 are generally referred to as side-effect tips. A side-effect tip may be defined as a tip that includes an active electrode with a surface disposed radially from a longitudinal axis of the rotation tube 54 (or the surgical device, generally). Tip 500 is generally referred to as a beveled tip, and may also be referred to as a side-effect tip. Tips 300 and 350, with electrodes at the end of the tips, may be particularly useful in knee surgery. Tips 300 and 350 are generally referred to as end-effect tips.

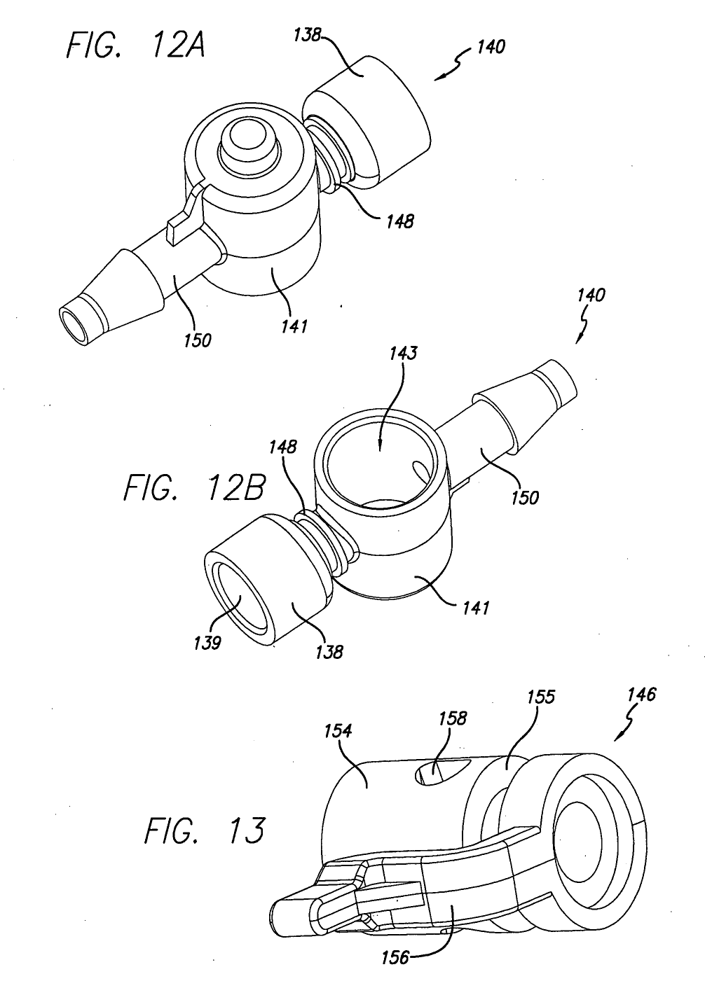

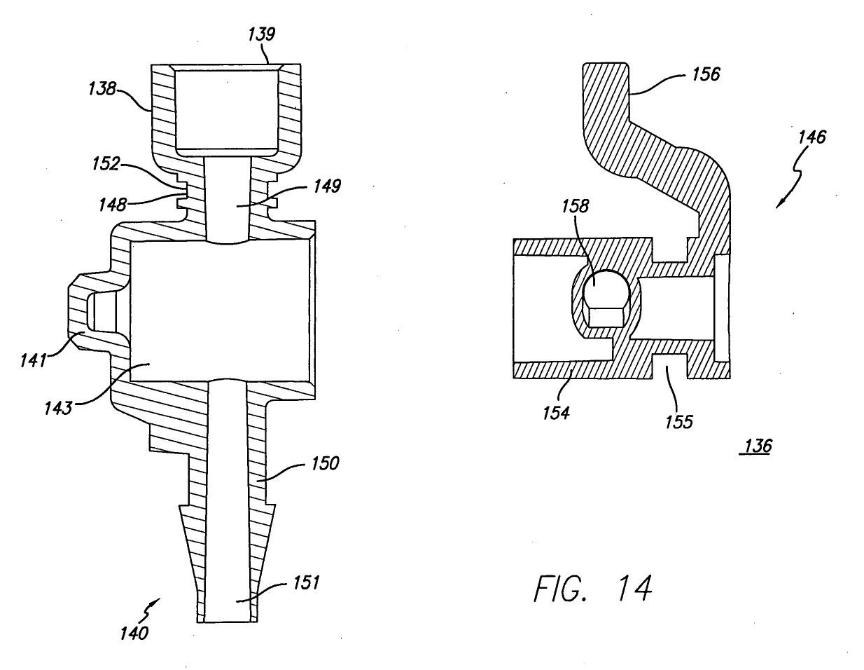

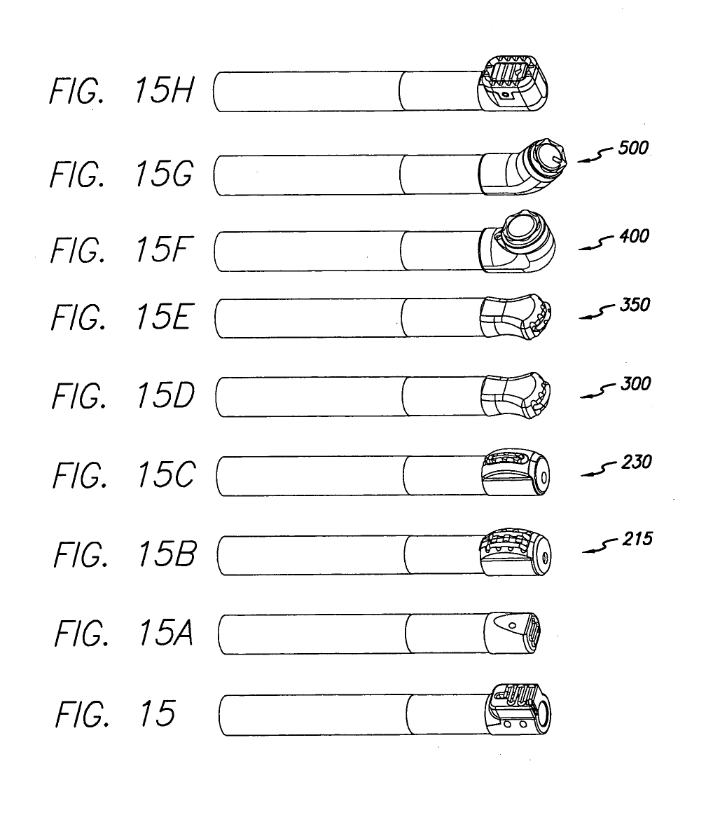

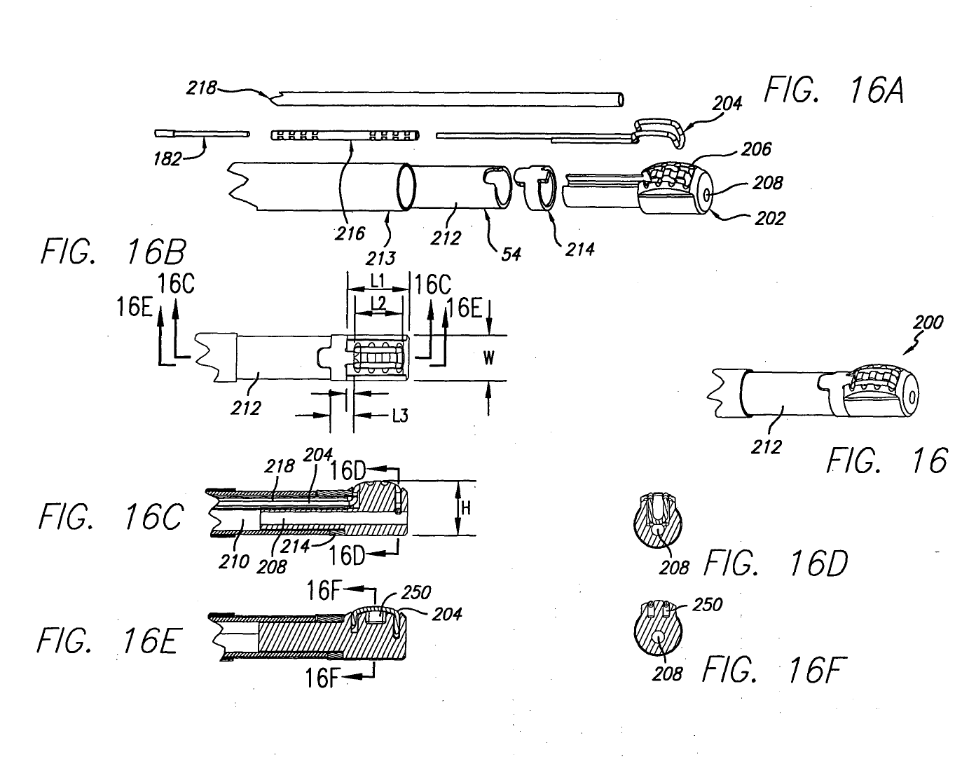

Referring to FIGS. 16-16F, a surgical tip 200 includes an electrically insulating, ceramic housing 202 and a formed wire electrode 204. Housing 202 includes a grooved and notched portion 206 and an aspiration lumen 208. Portion 206 is configured to engage with electrode 204 and to provide a textured surface having a formation that can be used, for example, to rasp tissue during use. Aspiration lumen 208 is in fluid communication with a lumen 210 defined by rotation tube 54 (FIG. 16C). Housing 202 is also configured to connect to an uninsulated portion 212 of rotation tube 54, i.e., the return electrode. An insulated portion 213 is insulated with a shrink polyester insulator. Housing 202 and rotation tube 54 can be connected, e.g., by a ceramic adhesive. Housing 202 and rotation tube 54 are joined by a ceramic collar 214, which acts as a spacer between the return electrode and electrode 204, e.g., to minimize the possibility of arcing. In some embodiments, collar 214 and housing 202 can be integrally formed as one member.

Electrode 204 is formed to engage with portion 206 of housing 202. At one end, electrode 204 is connected to active conductor 182 by a stainless steel crimp connector 216. The other end of electrode 204 terminates within and is surrounded by housing 202 to prevent a short circuit, e.g., if electrode 204 were to contact rotation tube 54. A polyimide insulator 218 insulates active conductor 182, crimp connector 216 and portions of electrode 204 (FIG. 16A). Electrode 204 is formed of tungsten wire and has a racetrack shaped loop with downwardly bent portions. At its distal end, electrode 204 curves down such that it is in fluid communication with lumen 208 (FIGS. 16C and 16D). As shown in FIGS. 16E and 16F, there are two cavities 250 in the surgical tip, one cavity 250 below each of the arms of electrode 204. Surgical tip 200 is sized to be received within a joint and housing 202 has a length, L1, of about 0.2 inches, a width, W, of about 0.142 inches, and a height, H, of about 0.171 inches. Further, the exposed electrode wires have a length, L2, of about 0.153 inches, and are separated from return 212 by a length, L3, of about 0.075 inches.

Referring to FIGS. 16G-16K, a surgical tip 215, which is similar to tip 200, has no collar 214 and has a pin 220. Pin 220 can be used to secure electrode 204 in place (FIG. 16J).

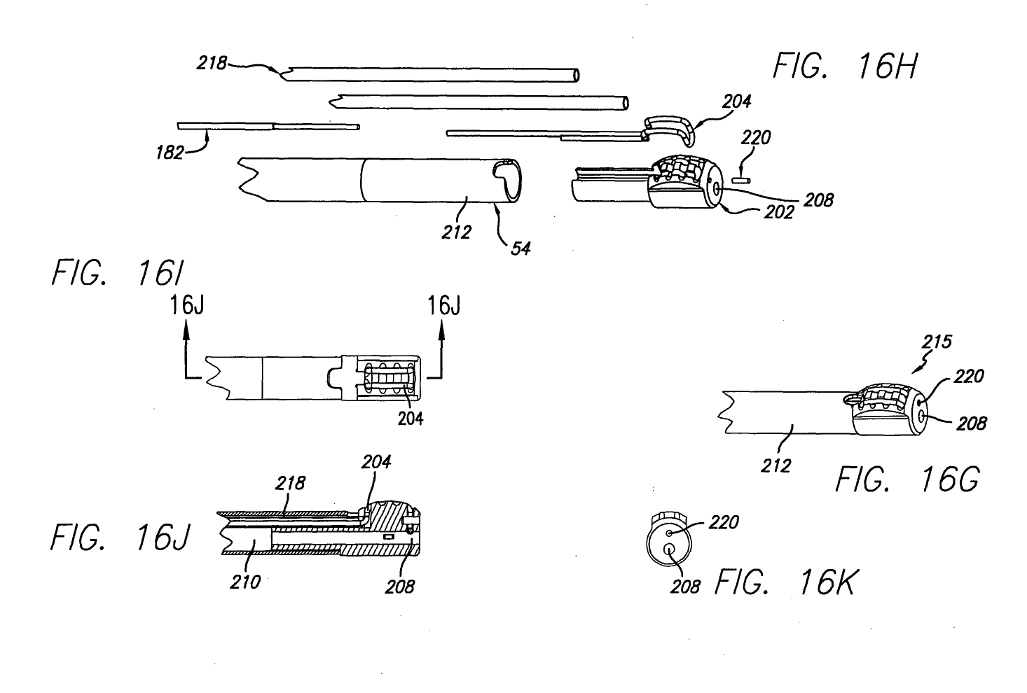

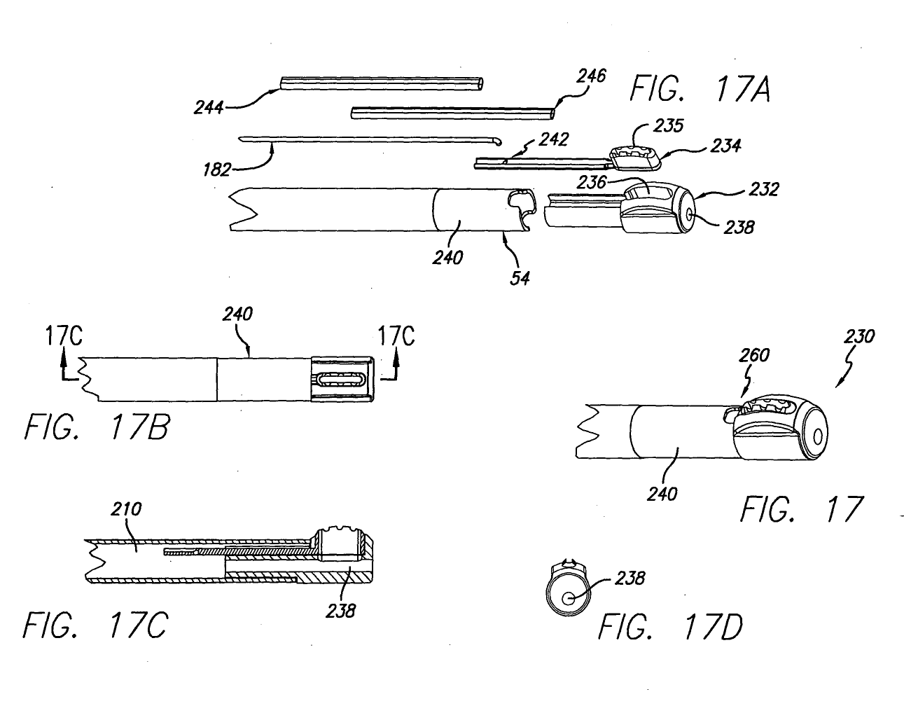

Referring to FIGS. 17-17D, a surgical tip 230 includes an electrically insulating, ceramic housing 232 and a tungsten electrode 234 formed by metal injection molding. Housing 232 includes a recessed portion 236 and an aspiration lumen 238. Recessed portion 236 is configured to receive electrode 234. Aspiration lumen 238 is in fluid communication with lumen 210 defined by rotation tube 54 (FIG. 17C). Housing 232 is also configured to engage with an uninsulated portion 240 of rotation tube 54, i.e., the return electrode. Return electrode 240 may contain one or more cut-outs 260.

Electrode 234 is formed to engage with recessed portion 236. Electrode 234 is formed with a sharp edge 235 that defines sharp ridges and/or grooves. The ridges and/or grooves are formations that help to create higher field intensities during use and can be used, for example, to rasp tissue during use. Electrode 234 is connected to active conductor 182 by engaging active conductor 182 to an opening 242 defined by the electrode. Active conductor 182 is surrounded by an insulator 244, e.g., a shrink polyester, and portions of the active conductor and electrode 234 are surrounded by an insulator 246, e.g., a polyimide.

Referring to FIGS. 18-18E, a surgical tip 300 includes an electrically insulating, ceramic housing 302 and a formed tungsten wire electrode 304. Housing 302 includes a grooved and notched distal end 306 with a groove 308 configured to receive electrode 304. The textured surface of distal end 306 provides formations that can be used, for example, to rasp tissue during use. The formations can be described as ridges or scallops, and have a curved top surface when viewed from the distal end. Groove 308 is in fluid communication with a suction tube 312. At its proximal end, suction tube 312 is in fluid communication with suction tubing 46. The thickness of groove 308 and the inner diameter of tube 312 are larger than the width of electrode 304 to provide a suction path into suction tube 312. Housing 302 is also configured to engage with an uninsulated portion 212 of rotation tube 54, i.e., the return electrode. In other implementations, tube 312 may be omitted or altered, using the lumen defined by rotation tube 54 and/or the pathway defined by groove 308 for suction, or eliminating suction altogether.

Electrode 304 is formed to fit in groove 308 of housing 302. At one end, electrode 304 is connected to active electrode 182, e.g., by soldering, mechanically crimping, etc. The other end of electrode 304 is separated from the first end of the electrode by tube 312. A shrink polyester insulator 314 surrounds active electrode 182, and a polyimide insulator 316 surrounds portions of the active conductor and electrode 304. Surgical tip 300 is sized to be received within a joint and housing 302 has a length, L1, of about 0.228 inches, a width, W, of about 0.166 inches, and a height, H, of about 0.092 inches. Further, to enable electrode 304 to contact tissue, electrode 304 extends beyond housing 302 by a length, L2, of about 0.009 inches.

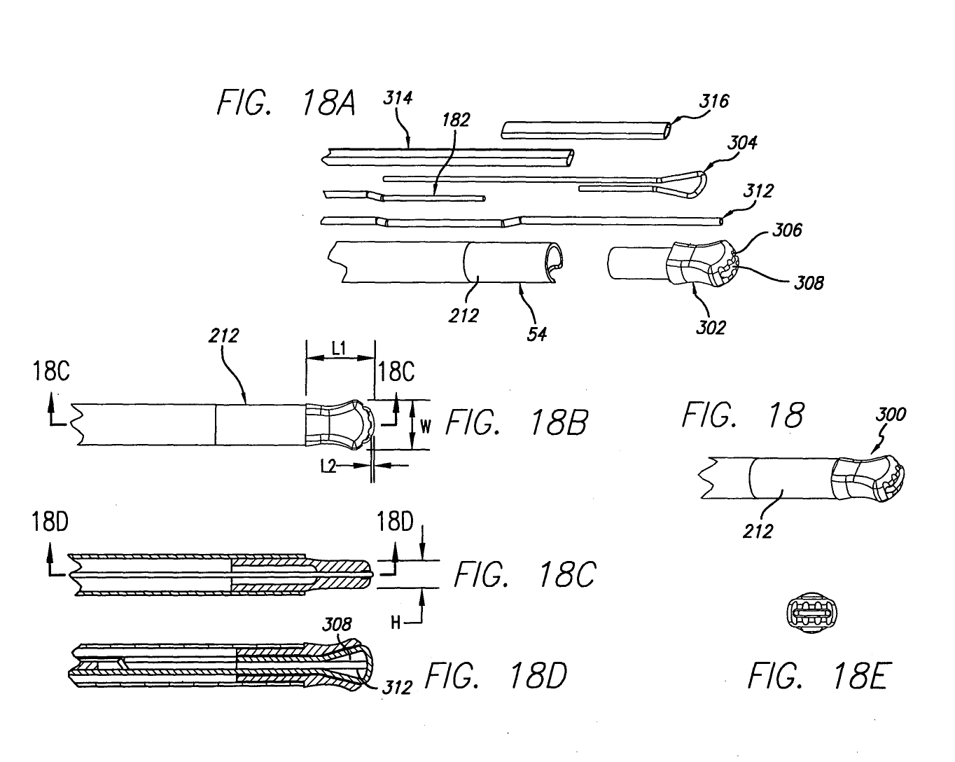

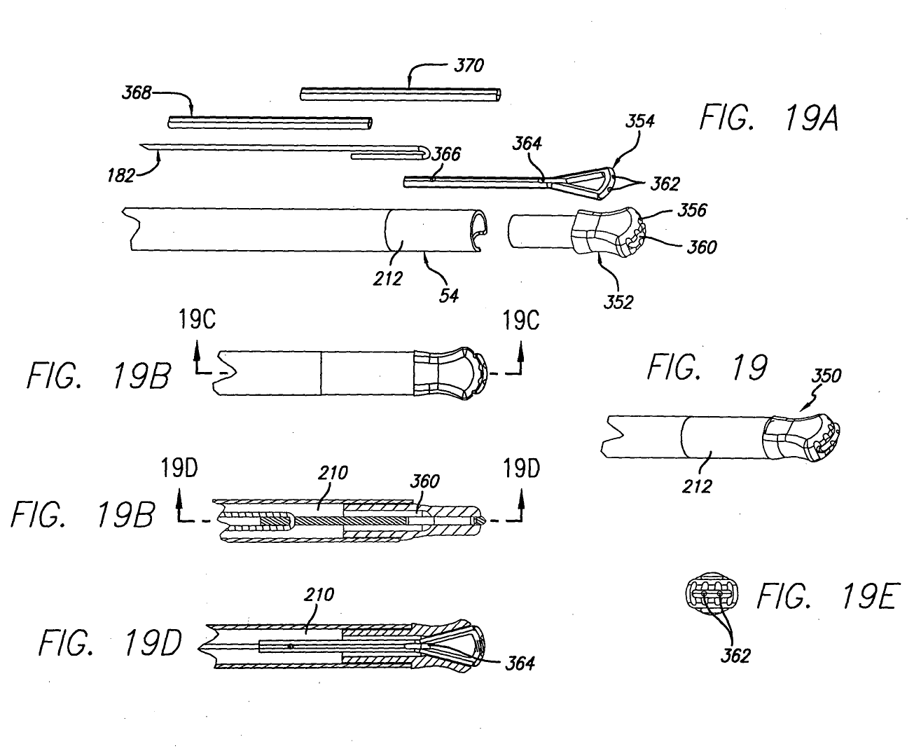

Referring to FIGS. 19-19E, a surgical tip 350 includes an electrically insulating, ceramic housing 352 and a tungsten electrode 354 formed by metal injection molding. Housing 352 includes a grooved and notched distal end 356. The textured surface of distal end 356 provides formations that can be used, for example, to rasp tissue during use. Housing 352 further defines an aspiration lumen 360 that is in fluid communication with a lumen 210 defined by rotation tube 54. Housing 352 is also configured to engage with an uninsulated portion 212 of rotation tube 54, i.e., the return electrode.

Electrode 354 is configured to engage with and fit inside aspiration lumen 360. Electrode 354 defines openings 362 that are in fluid communication with lumen 210 defined by rotation tube 54 to provide an aspiration path to suction tube 46. During aspiration, aspirated material flows through openings 362, pass recessed portions 364 defined by electrode 354, and into lumen 210. At its proximal end, electrode 354 is connected to active conductor 182 by hooking the active conductor through an opening 366 defined by the electrode. A shrink polyester insulator 368 surrounds active electrode 182, and a polyimide insulator 370 surrounds portions of the active conductor and electrode 354.

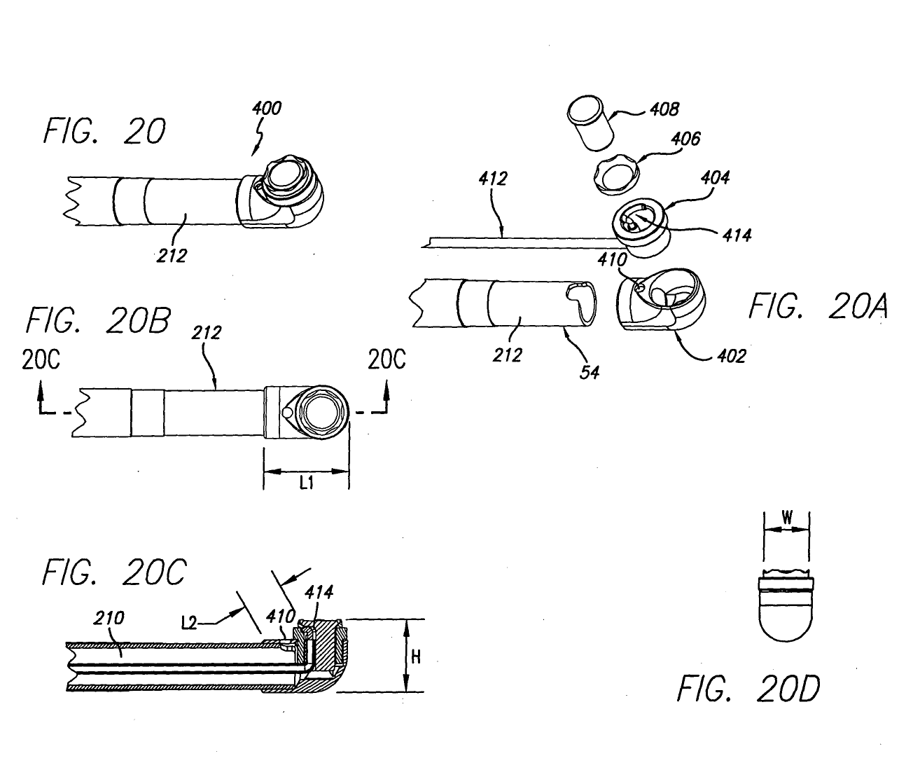

Referring to FIGS. 20-20D, a surgical tip 400 includes a housing 402, a thermal band 404, an active electrode 406, e.g., tungsten, and an electrically insulating ceramic thermal pin 408. Housing 402 is formed of an electrically conducting material, e.g., stainless steel, and is configured to engage with an uninsulated portion 212 of rotation tube 54. Thus, in this embodiment, housing 402 and portion 212 act as the return electrode. Housing 402 also defines an aspiration opening 410 that is in fluid communication with lumen 210 defined by rotation tube 54. Surgical tip 400 is sized to be received within a joint and housing 402 has a length, L1, of about 0.259 inches, electrode 406 has a width, W, of about 0.135 inches, and tip 400 has a height, H, of about 0.217 inches. Further, to provide a bipolar path, electrode 406 is separated from return 212 by a length, L2, of about 0.121 inches.

Thermal band 404 is made of an electrically insulating material, e.g., a ceramic, and is disposed in housing 402. Active conductor 182 (not shown), which is surrounded by a polyimide insulator 412, extends along rotation tube 54 and up into thermal band 404. An uninsulated portion 414, e.g., bare copper wire, of active conductor 182 is fitted into a recess defined by thermal band 404.

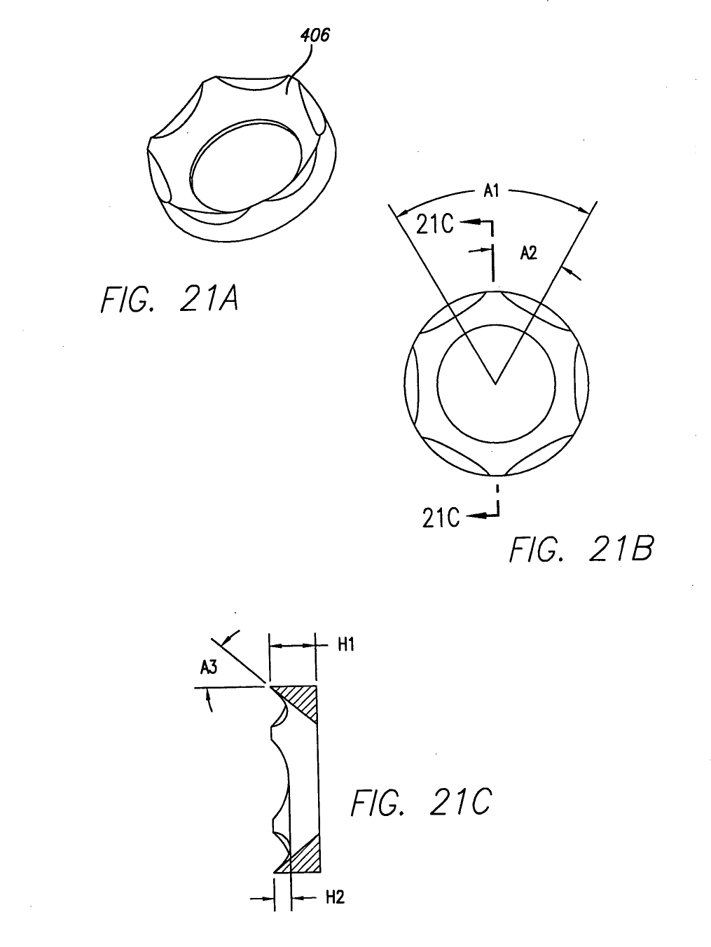

Electrode 406 is a ring-shaped member having a top circumference with ridges and grooves, e.g., like the top of a rook piece in chess. The textured top surface of electrode 406 provides formations that can be used, for example, to rasp tissue during use. Referring to FIGS. 21A-C, detailed views of electrode 406 include illustrative dimensions. Electrode 406 is sized to be received within housing 402 and has a height, H1, of about 0.025 inches. Electrode 406 is designed to provide points of plasma generation and has a height, H2, of about 0.01 inches, an angle, A1, of about sixty degrees, an angle, A2, of about thirty degrees, and an angle, A3, of about forty degrees.

When assembled, thermal pin 408 and electrode 406 engage thermal band 404, and a bottom portion of electrode 406 contacts portion 414 (FIG. 20C). To accommodate active conductor 182, thermal pin 408 includes a cut away portion 414 that receives the active conductor (FIG. 20C).

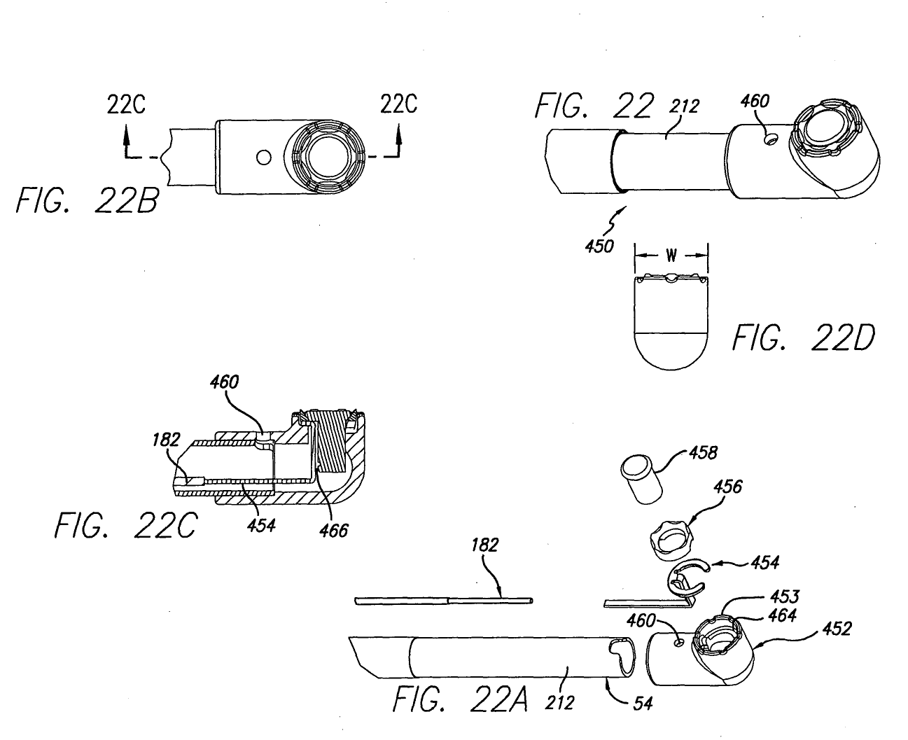

Referring to FIGS. 22-22D, a surgical tip 450 includes an electrically insulating, ceramic housing 452, an electrically conducting, e.g., stainless steel, connector 454, an active electrode 456, e.g., tungsten, and an electrically insulating, ceramic thermal pin 458. Housing 452 is configured to engage an uninsulated portion 212 of rotation tube 54, i.e., the return electrode. Housing 452 includes an aspiration opening 460 that is in fluid communication with lumen 210 defined by rotation tube 54. Housing 452 also defines a top circumference 453 with ridges and notches that are formations that can be used, for example, to rasp tissue. The ridges on housing top surface 453 have a flat top, where the top is defined as in FIG. 22B. The formation of the top surface of electrode 456 can also be used to rasp tissue during use. Surgical tip 450 is sized substantially the same as surgical tip 400 in FIGS. 20-20D.

At its distal end, connector 454 defines a horseshoe-shaped portion 462 that rests on a surface 464 defined by housing 452 when electrode 450 is fully assembled. At its proximal end, connector 454 is connected to active conductor 182. Portions of connector 454 and active conductor 182 within rotation tube 54 are electrically insulated, e.g., with a polyimide insulator as described above.

Electrode 456 and thermal pin 458 are generally similar to electrode 406 and thermal pin 408, respectively. When assembled, thermal pin 458 and electrode 456 engage with housing 452, with a bottom portion of electrode 456 making good contact with connector 454 (FIG. 22C). To accommodate for connector 454, thermal pin 458 defines a cut away portion 466 that receives the connector (FIG. 22C).

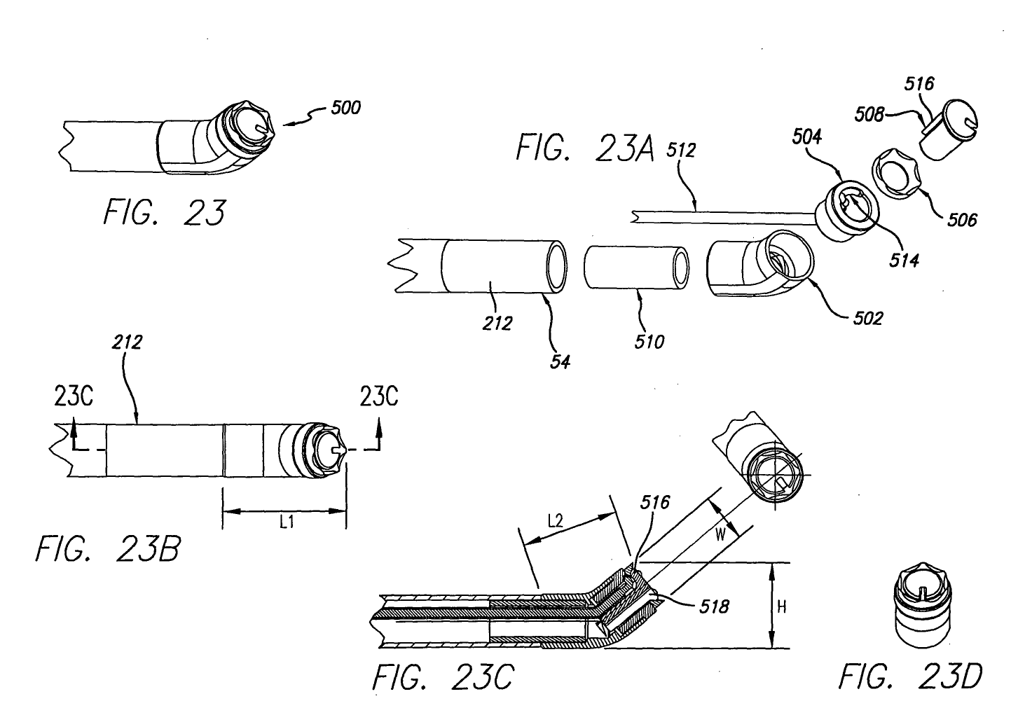

Referring to FIGS. 23-23D, a surgical tip 500 is similar, though not identical, to tip 400. Tip 400 is angled about ninety degrees relative to the length of rotation tube 54, whereas tip 500 is positioned at a non-ninety degree angle relative to the length of the rotation tube.

Tip 500 generally includes an electrically conducting housing 502, e.g., stainless steel, an electrically insulating, e.g., ceramic, thermal band 504, an active, e.g., tungsten, electrode 508, and an electrically insulating, e.g., ceramic, thermal pin 508. Housing 502 is configured to engage with an uninsulated portion 212 of rotation tube 54 by a conductive, e.g., stainless steel, coupler 510. In some embodiments, housing 502 and coupler 510 are integrally formed as one member.

Thermal band 504 is configured to be disposed in housing 402. Active conductor 182, which is surrounded by a polyimide insulator 512, extends along rotation tube 54 and up into thermal band 504. An uninsulated portion 514, e.g., bare copper wire, of active conductor 182 is fitted into a recess defined by thermal band 404. Surgical tip 500 is sized to be received within a joint and has a length, L1, of about 0.32 inches, a width, W, of about 0.128 inches, and a height, H, of about 0.222 inches. Further, to provide a bipolar path, electrode 506 is separated from return 212 by a length, L2, of about 0.252 inches.

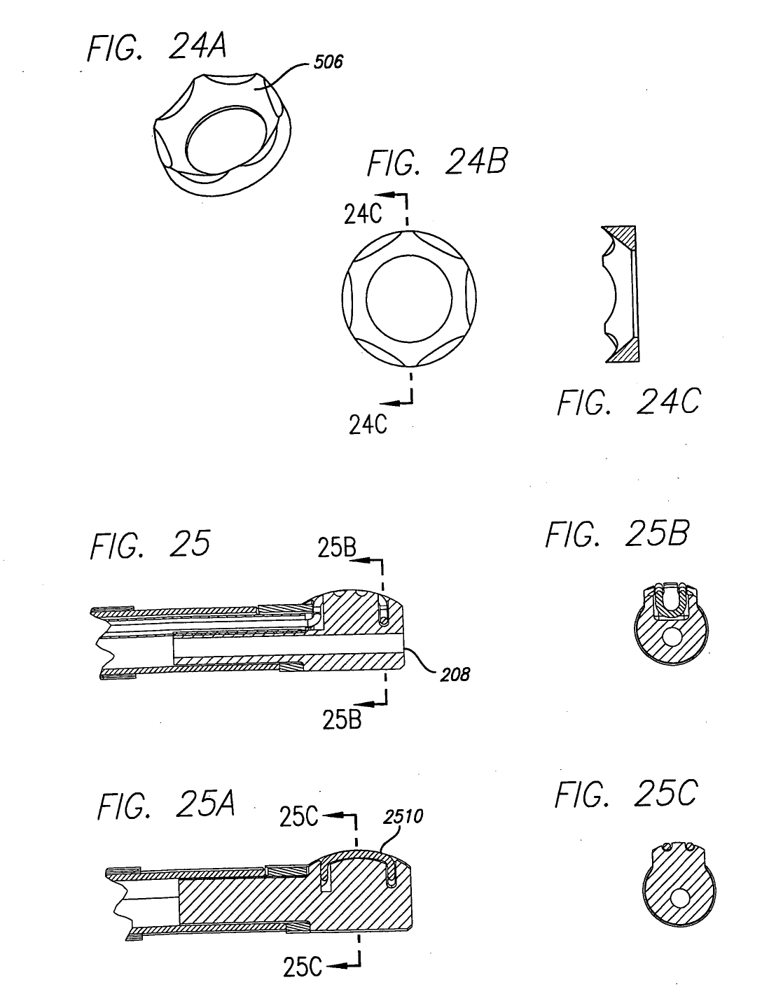

Electrode 506 is a ring-shaped member having a top circumference with ridges and grooves, e.g., like the top of a rook in chess, which can be referred to as castleations. The textured top surface of electrode 506 provides formations that can be used, for example, to rasp tissue during use. FIGS. 24A-24C show detailed views of electrode 506. The dimensions are substantially similar to those in FIGS. 21B and 21C.

When assembled, thermal pin 508 and electrode 506 engage with thermal band 504, and a bottom portion of electrode 506 contacts portion 514 (FIG. 23C). To accommodate for active conductor 182, thermal pin 508 defines a cut away portion 516 that receives the active conductor (FIG. 23C). Thermal pin 508 also defines an aspiration lumen 518 that is in fluid communication with lumen 210 defined by rotation tube 54.

Referring to FIGS. 25-25C, rather than electrode 204 penetrating the aspiration lumen 208 (FIG. 16C), an electrode 2510 does not protrude into suction lumen 208. Further, there are no cavities 250 below the arms of electrode 2510 (compare FIGS. 16E-16F with FIGS. 25A and 25C).

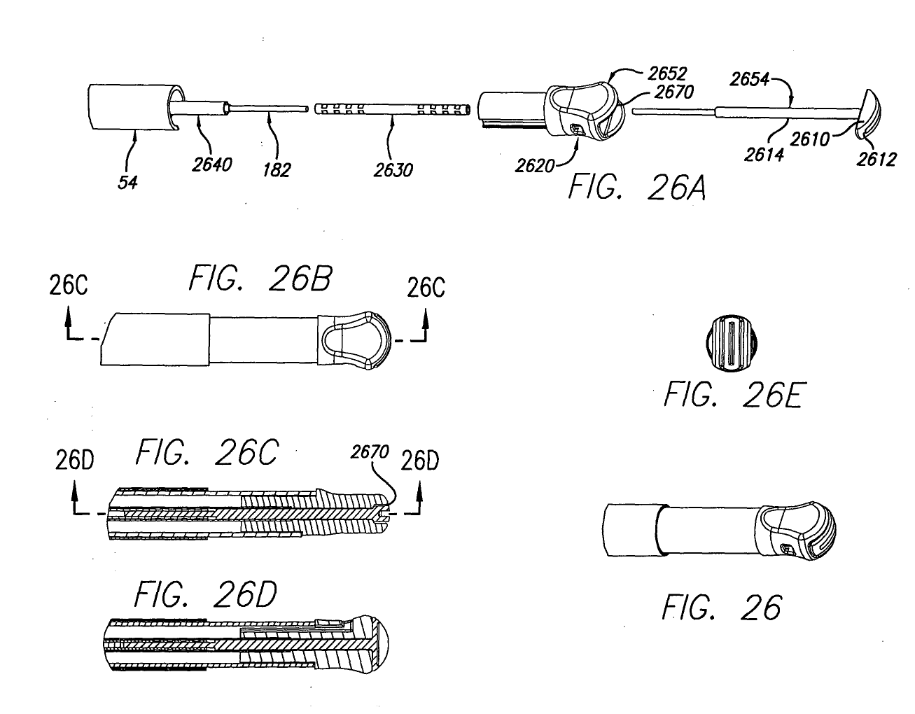

Referring to FIGS. 26-26E, an electrode 2654 has a different shape than electrode 354 of FIG. 19A. Electrode 2654 can be metal injection molded and includes a distal tip 2610 with a groove 2612 that is a formation that can be used for rasping, and includes a proximal end 2614. A housing 2652 has a different surface contour at the distal end than housing 352 of FIG. 19A. Housing 2652 has a formation 2670 that can be described as a groove, or as a ridge or an edge, and that provides rasping capability. Electrode 2654 does not define a suction lumen, in contrast to electrode 354 of FIG. 19A. Rather, suction is provided through a suction hole 2620 in a side of housing 2652. Suction hole 2620 is in fluid communication with the interior of rotation tube 54 and proximal end 2614 of the electrode may be off-center to accommodate the fluid communication and/or desired wall thicknesses.

Further, electrode 2654 connects to copper wire 182 using a crimp connector 2630, rather than folding over wire 182 as in FIG. 19A. Crimp connector 2630 is mechanically crimped to both electrode 2654 and copper wire 182. A polyimide insulator 2640 covers wire 182, the crimp connector 2630, and an exposed portion of electrode 2654. Polyimide insulator 2640 can be inserted into housing 2652, as shown in FIGS. 26C-26D. Polyimide insulator 2640 can be further secured in housing 2652 by using an epoxy, for example a ceramic-based epoxy. An epoxy can be used to secure housing 2652 to rotation tube 54.

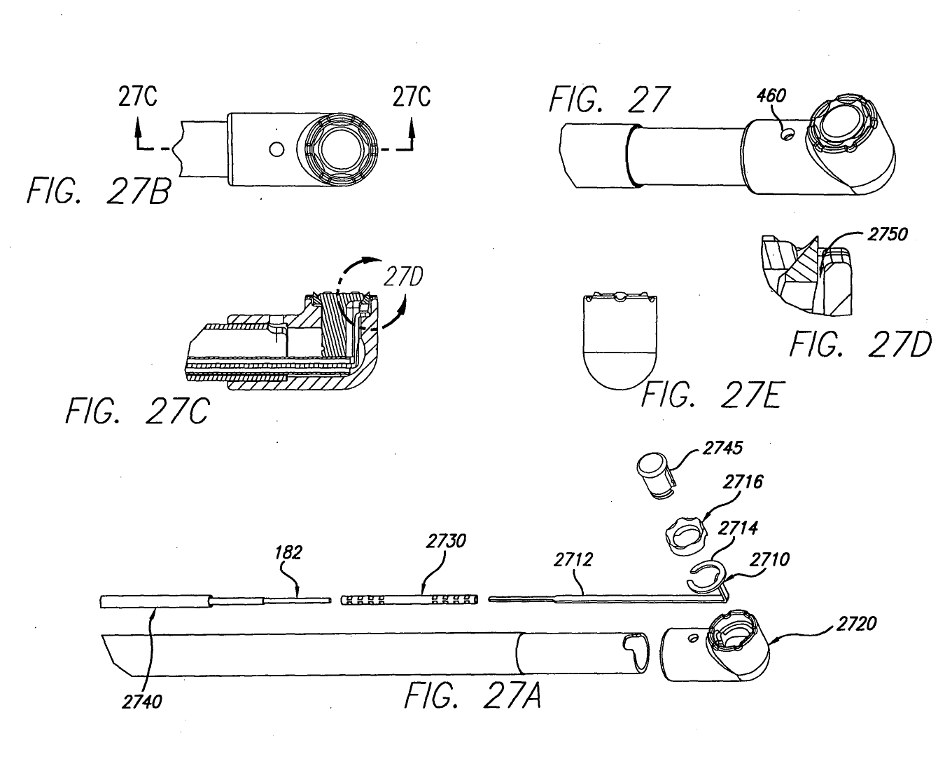

Referring to FIGS. 27-27E, a connector 2710 can be made from phosphor bronze, which may be a better conductor than the stainless steel used for connector 454 in FIG. 22A. Further, connector 2710 includes a lead 2712 that connects to a distal end of a contact surface 2714. Contact surface 2714 may contact an electrode 2716. Lead 2712 makes an approximately ninety degree turn toward electrode 2716 near the bottom of a housing 2720. Lead 2712 thus provides more clearance for suction hole 460 than that shown in FIG. 22C.

Connector 2710 is connected to wire 182 using a crimp connector 2730 made of stainless steel. A polyimide insulator 2740 may be used to insulate all or part of wire 182, crimp 2730, and lead 2712. As shown in FIG. 27C, insulator 2740 may cover lead 2712 up to the point where lead 2712 turns toward contact surface 2714. An epoxy may also be used to retain connector 2710 and/or a thermal pin 2745 in place, and the epoxy may be applied, for example, distally up to the point where lead 2712 turns toward contact surface 2714. FIG. 27D illustrates a particular implementation in which epoxy does not completely surround, that is, encircle the outer perimeter of, electrode 2716, as indicated by reference numeral 2750.

Dimensions in the embodiment of FIGS. 27-27E are substantially similar to the dimensions in FIGS. 20-20D and 22-22D. It can also be seen that the raised edges of electrode 2716 align with the low points of housing 2720, in contrast to FIGS. 22-22D in which the raised portions of electrode 456 align with raised portions of housing 452.

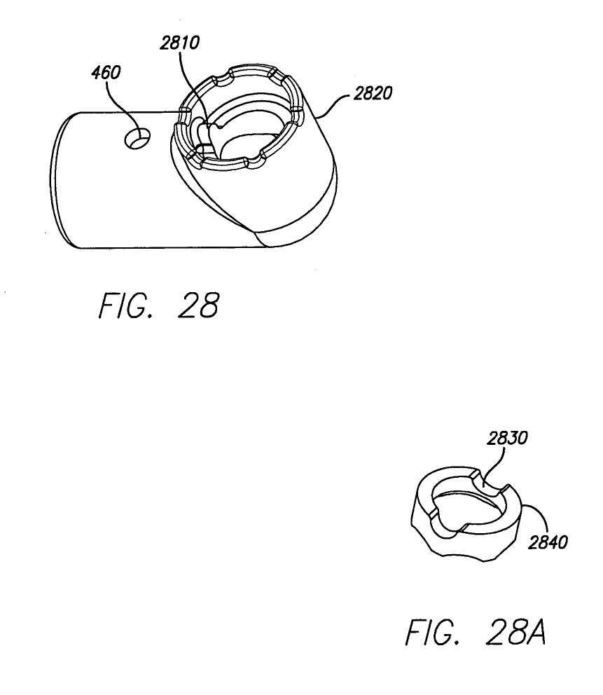

Referring to FIGS. 28-28A, a keying tab 2810 is highlighted on a housing 2820 (see also FIG. 27A) for aligning an electrode. Keying tab 2810 may also align a connector (see connector 2710 in FIG. 27A). In housing 2820, suction hole 460 is closer to the bend in the housing, as compared to housing 2720 in FIG. 27A. FIG. 28A shows female key slots 2830 on the bottom of an electrode 2840.

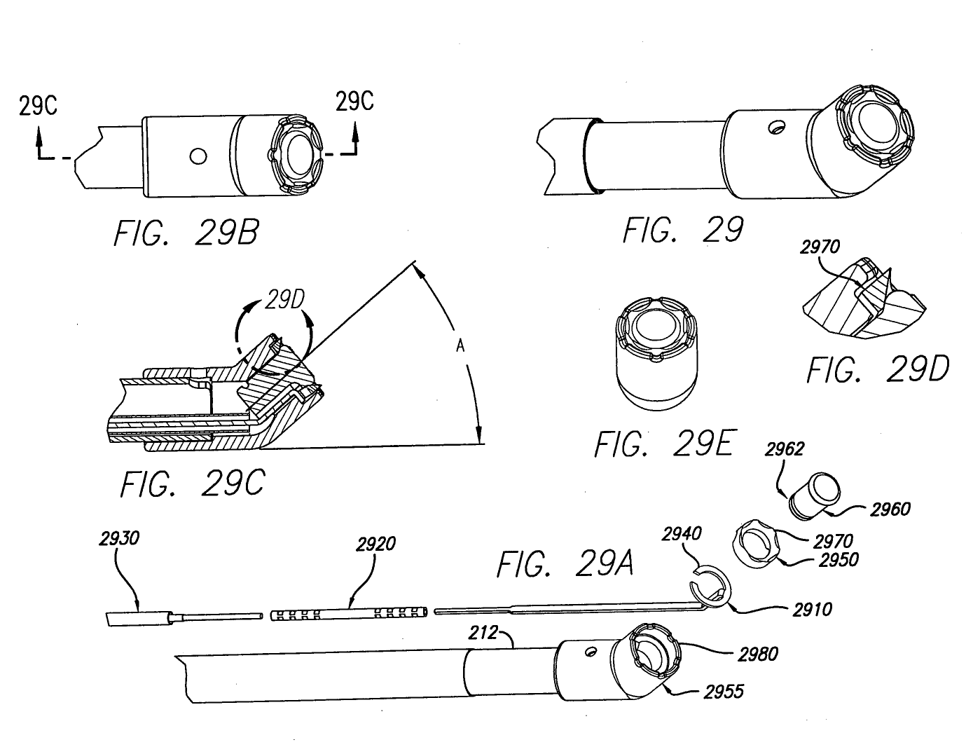

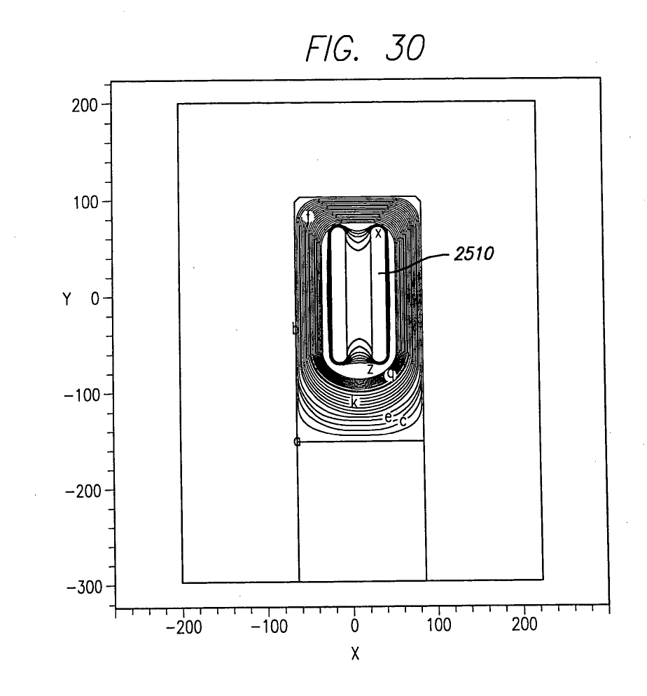

Referring to FIGS. 29-29E, a connector 2910 is configured substantially similarly to connector 2710 in FIGS. 27-27E, including the use of a crimp connector 2920 and a polyimide insulator 2930. Connector 2910 provides a contact surface 2940 for contacting an electrode 2950. Contact surface 2940 forms substantially a complete circle, providing almost three-hundred sixty degrees of contact. This is more than that provided in FIG. 23A by wire 514 contacting electrode 506 over approximately a ninety degree portion of a circle.

As described for FIGS. 26-26E and FIGS. 27-27E, an epoxy may be used to secure connector 2910 to a housing 2955. In a particular implementation, the epoxy is applied distally until is contacts a thermal pin 2960 and forms around an indented groove 2962 near the base of pin 2960. In that implementation, the epoxy may wick up part of the outside surface of pin 2960, but stops short of completely surrounding electrode 2950, as shown by reference numeral 2970 in FIG. 29D. In one implementation, thermal pin 2960 is approximately 0.145 inches in length, the length being associated with the longest dimension.

Electrode 2950 is similar to electrodes 2716, 2840 in FIGS. 27-27E and FIG. 28A, and includes key slots on its bottom surface that align electrode 2950 in housing 2955. The top surface of electrode 2950 is designed to provide high points 2970 at specified angles with respect to the geometry of scallops 2980 on housing 2955 and with respect to a return electrode 212. In the embodiment of FIGS. 29-29E, high points 2970 occur at approximately sixty degree intervals and align with the low points of scallops 2980, and the shortest distance between electrode 2950 and return electrode 212 is L1, which is about 0.309 inches.

High points 2970 may provide areas of higher current density, also referred to as concentrations of current density. The concentrations of current density facilitate creation of a vapor barrier and plasma generation from one or more points 2970 on electrode 2950. The generation of a plasma is commonly referred to as light off. The electrodes of FIGS. 20-20D, 21A-C, 22-22D, 23-23D, 24A-C, 27-27E, 28A, and 29-29E include multiple high points that may each provide a location for light off. The other disclosed electrodes may also provide light off from various locations along the electrode depending on the design. Scallops 2980, more particularly referred to as castleations, are utilized in several of the embodiments in this disclosure and are features that provide rasping capability. The embodiment of FIGS. 29-29E is sized to be received in a joint and the dimensions are substantially similar to previous embodiments. The embodiment of FIGS. 29-29E is designed to have a beveled tip with an angle, A, of about forty degrees.

Referring to FIGS. 30-34, there are shown various results from a finite element analysis of the surgical tips depicted in FIGS. 25-25F, FIGS. 18-18E, and FIGS. 27-27E. The analysis models one or more electrical characteristics, such as, for example, electric field strength, voltage, current, or power, to determine probe configurations that provide desired design objectives. For example, design objectives can include, for a particular electrical characteristic, providing for (i) substantial uniformity around an electrode, (ii) a maximum value at a point above and to the outside of an electrode envelope, (iii) quick drop-off as a function of distance from an electrode, and (iv) quick drop-off as a function of tissue depth.

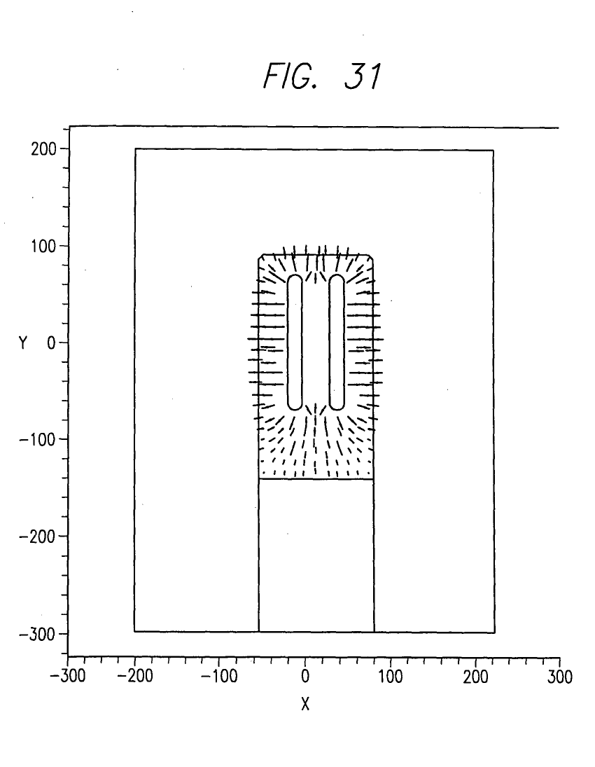

Referring to FIGS. 30-31, a model of the surgical tip of FIGS. 25-25F, shown as atop view, looking at the face of the surgical tip through tissue, assumes that the wires of electrode 2510 (FIGS. 25-25C) are buried in tissue to the surface of ceramic housing 202 (FIGS. 16-16F), which is approximately the surface of electrode 2510. The model also assumes that the surgical tip is immersed in a medical grade saline solution containing 0.9% saline. Thus, the region outside of the surgical tip is modeled as consisting of the saline solution. The plane of view can also be expressed in terms of an engagement angle. An engagement angle refers to the angle at which the surgical tip contacts tissue. In the present model, the engagement angle is perpendicular to the face of electrode 25.10.

The surgical tip of FIGS. 25-25F is shown superimposed with isometric lines of constant electric potential (voltage). The potential is substantially uniform around the entire envelope of the electrode. The envelope of the electrode refers to the smallest rectangle, or other closed shape, that will enclose the electrode in the plane being viewed. In this case, the envelope is the smallest rectangle that will enclose both wires of the electrode in the plane being viewed. This feature allows a surgeon to effectively operate on tissue by providing relatively uniform electrical characteristics around the entire perimeter of the electrode.

FIG. 30 also shows that the strength of the potential falls off to approximately half of its maximum value by 3/100 of an inch from the electrode surface around the entire periphery of the envelope. The maximum is achieved at the top right corner of the electrode, and the entire periphery of the electrode is at substantially the maximum value. When the electric field strength falls off quickly after the tissue surface, it reduces the risk of burning tissue below the surface tissue that is of interest.

Referring to FIG. 31, the electric field strength, measured in volts per thousandth of an inch (volts/mil), represents the gradient of the potential. The graph displays the electric field as a vector. The maximum electric field strength is outside of the envelope of the electrode, which facilitates operating on tissue by not having to center the tissue over the electrode in order to take advantage of the maximum electric field strength.

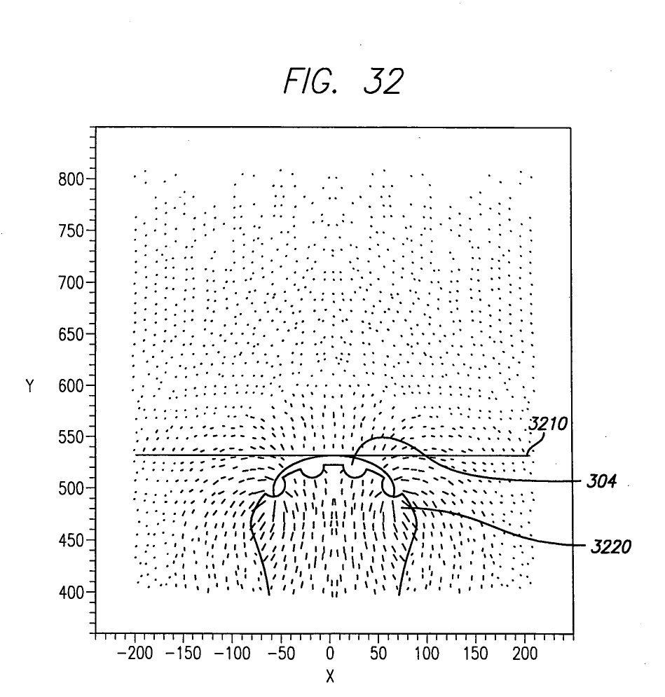

Referring to FIG. 32, a model of the surgical tip of FIGS. 18-18E is shown from a side view along a longitudinal cross-section down the middle of electrode wire 304. The model assumes that electrode 304 is touching the tissue, indicated by a solid horizontal line 3210. The model further assumes that the region below the tissue and outside of the surgical tip is the medical grade saline solution. The electric field strength at the tissue surface has dropped by more than 65% from a maximum value 3220. Within 3/100 of an inch into the tissue, the strength of the electric field has fallen by more than 50% from the strength at the tissue surface and by more than 85% from the maximum value. The envelope of electrode 304 can be taken to be a rectangle having an upper edge at the line representing the tissue surface, and having two side edges coming down from the upper edge at approximately +/−60 mils on the x axis.

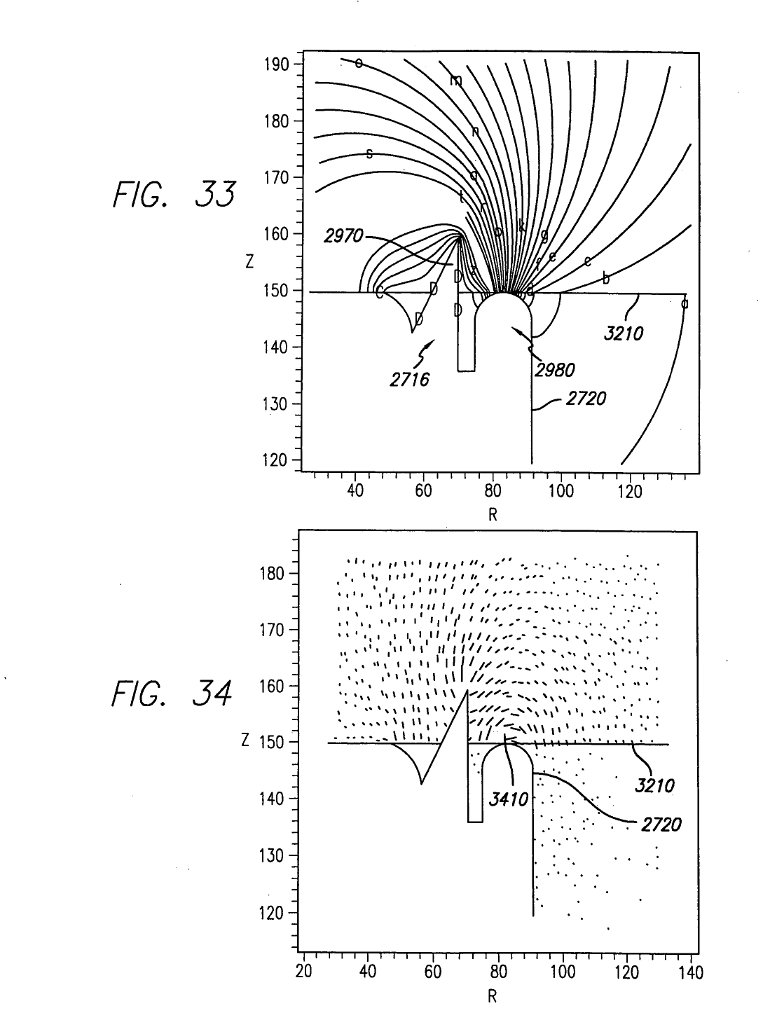

Referring to FIGS. 33-34, a model of the surgical tip of FIGS. 27-27E is shown from a side view along a longitudinal cross-section down the middle of the surgical tip, similar to the view depicted in FIG. 27C. As indicated in FIG. 27B, the cross-section goes through a high point (2970 in FIG. 29A) of electrode 2716, and through a low point on one of the scallops (2980 in FIG. 29A) on housing 2720. In the model, the high point of the electrode is assumed to have penetrated tissue surface 3210 by approximately ten mils. The model further assumes that the region below the tissue and outside of the surgical tip is the medical grade saline solution.

Referring to FIG. 33, at a tissue depth of approximately 30 mils, the potential has dropped by more than 40% from its maximum, which occurs along the surface of the high point that is labeled as “D.” At a tissue depth that is approximately 30 mils deeper than the high point, the potential has dropped by more than 45%, or almost half, from its maximum.

Referring to FIG. 34, the electric field strength at a tissue depth of approximately 15 mils has fallen by more than 50% from a maximum 3410, which occurs just above housing 2720. The electric field strength at a tissue depth of approximately 30 mils has fallen by more than 70% from its maximum. Maximum value 3410 occurs at a position that is above substantially all of the electrode, and at points above the high point, the electric field strength is at least approximately 70% of the maximum value. Being “above” the electrode refers to being away from the electrode surface in a favorable direction for contacting tissue. The electrode envelope extends from the left side of the graph to the right up to the edge of the electrode, which is at approximately 68 mils on the x axis.

Modifications to the disclosed implementations can be made. For example, the features described for one or more of the disclosed surgical tips can generally be applied to other disclosed tips. Such features include, for example, electrode geometry and materials, housing geometry and materials, and aspiration techniques. For example, in some embodiments, probe 36 does not include a suction feature. As a further example, any of the disclosed tips may include one or more surfaces that have a formation for providing a mechanical rasping action against tissue.

Such rasping action may be provided, for example, by a housing or an electrode. The housing or electrode may have a formation such as, for example, an elevated or depressed area, such as a deposit or pit, arising from, for example, (i) a manufacturing process using, for example, a mold, (ii) a chemical process that may etch a surface or leave a deposit, (iii) a coating or the addition of another material or object to the housing or electrode, or (iv) a mechanical process such as, for example, sanding or scraping. A formation may also include, for example, (i) an edge, (ii) a point, (iii) a groove, (iv) a ridge, (v) a scallop, (vi) a castleation, (vii) some other area of raised elevation with respect to another surface, (viii) a non-smooth surface contour, (ix) a surface roughened by, for example, a chemical or mechanical process, or (x) some other surface feature useful for rasping.

The disclosed materials are only examples and other suitable materials may be used. For example, implementations may use an insulator that is not a polyimide and a housing that is not a ceramic. Insulating portions may also include an electrically non-conductive, refractory material.

A number of implementations have been described. Nevertheless, it will be understood that various modifications can be made. Accordingly, other implementations are within the scope of the following claims.

What is claimed is:

1. A surgical device comprising:
an active electrode; and
an insulating region adjacent the active electrode, the insulating region having a surface with a formation for providing a mechanical rasping action against tissue.

2. The surgical device of claim 1 wherein the formation comprises a groove.

3. The surgical device of claim 1 wherein the formation comprises a ridge.

4. The surgical device of claim 3 wherein the ridge has a flat top-surface.

5. The surgical device of claim 3 wherein the ridge has a curved top-surface.

6. The surgical device of claim 1 wherein the formation comprises a feature selected from a group consisting of a scallop, an edge, and a point.

7. The surgical device of claim 1 wherein the insulating region substantially encircles a periphery of the active electrode.

8. The surgical device of claim 1 wherein the insulating region comprises an electrically non-conductive, refractory material.

9. The surgical device of claim 1 wherein the active electrode includes a configuration that concentrates current density.

10. The surgical device of claim 9 wherein the configuration comprises a raised portion.

11. The surgical device of claim 1 further comprising:
a hand wand; and
a shaft coupled to the hand wand for rotation relative to the hand wand, the shaft including the active electrode and the insulating region.

12. The surgical device of claim 11 wherein the shaft is continuously rotatable, such that the active electrode is continuously rotatable.

13. The surgical device of claim 11 wherein the shaft defines an aspiration lumen.

14. The surgical device of claim 13 further comprising:
a tube coupled to the shaft, the tube defining a lumen in communication with the aspiration lumen; and
a control coupled to the tube for controlling suction through the aspiration lumen.

15. The surgical device of claim 14 wherein the control comprises a valve.

16. The surgical device of claim 11 further comprising a control coupled to the shaft for rotating the shaft.

17. The surgical device of claim 16 wherein the control comprises a hand-actuated knob.

18. The surgical device of claim 11 further comprising a control coupled to the hand wand for controlling power applied to the active electrode.

19. The surgical device of claim 18 wherein the control comprises a push button.

20. The surgical device of claim 1 wherein an electrical characteristic of the surgical device is substantially uniform around a periphery of the active electrode when the electrical characteristic is measured in a plane, the plane being perpendicular to an engagement angle between the active electrode and a tissue surface, and the plane going through part of the active electrode.

21. The surgical device of claim 20 wherein:
the electrical characteristic comprises electric field strength,
the engagement angle comprises an angle providing substantially maximum tissue contact between the active electrode and a flat tissue surface, and
the active electrode comprises a surface configured to contact tissue at an angle that is not parallel to a longitudinal axis of the surgical device.

22. The surgical device of claim 1 wherein:
the active electrode defines an envelope in a given plane, the given plane going through the active electrode, and
an electrical characteristic of the surgical device measured at any point in the given plane that is at least 3/100 of an inch outside of the envelope drops off to no more than 60% of a maximum value for the electrical characteristic in the given plane.

23. The surgical device of claim 22 wherein:
the electrical characteristic comprises electric field strength,
the given plane is perpendicular to an engagement angle between the active electrode and a tissue surface, the engagement angle providing substantially maximum tissue contact between the active electrode and a flat tissue surface, and
the active electrode comprises a surface configured to contact tissue at an angle that is not parallel to a longitudinal axis of the surgical device.

24. The surgical device of claim 1 wherein:
the active electrode contacts a tissue surface,
a plane is defined going through the active electrode and the tissue surface, and
an electrical characteristic of the surgical device measured at any point in the plane corresponding to a tissue depth of at least 3/100 of an inch drops off to no more than 60% of a maximum value in the plane.

25. The surgical device of claim 24 wherein:
the electrical characteristic comprises electric field strength,
the electric field strength drops off to no more than half the maximum value at any point in the plane corresponding to a tissue depth of at least 15/1000 of an inch, and
the plane is parallel to an engagement angle between the active electrode and the tissue surface.

26. The surgical device of claim 1 wherein:
the active electrode defines an envelope in a given plane, the given plane going through the active electrode, and
an electrical characteristic of the surgical device achieves a maximum value in the given plane at a point outside of the envelope.

27. The surgical device of claim 1 further comprising a return electrode.

28. The surgical device of claim 27 further comprising a shaft, wherein the active and return electrodes are disposed on the shaft forming a bipolar surgical device.

29. The surgical device of claim 27 further comprising an adapter electrically coupled to the active electrode and the return electrode, the adapter being configured:
to couple to a generator,
to convert monopolar output from the generator into bipolar output, and
to couple the bipolar output to the active and return electrodes.

30. The surgical device of claim 27 wherein the adapter is further configured to convert substantially constant power output from the generator into substantially constant voltage output.

31. A method comprising:
applying electrical energy to tissue using an active electrode of a surgical device; and
rasping tissue mechanically using a formation on a surface of an insulating region, the insulating region being adjacent the active electrode.

32. The method of claim 31 wherein rasping tissue comprises using a ridge as the formation.

33. The method of claim 31 wherein applying electrical energy comprises concentrating current density with a configuration on the active electrode.

34. The method of claim 31 wherein rasping tissue comprises providing a user of the surgical device tactile feedback from tissue.

35. The method of claim 31 further comprising penetrating a joint in a body with the active electrode and the formation of the surgical device.

36. The method of claim 31 further comprising ablating tissue with the applied electrical energy.

37. The method of claim 31 further comprising coagulating tissue with the applied electrical energy.

38. A surgical device comprising:
an active electrode; and
an insulating region adjacent the active electrode, the insulating region having a surface adapted for providing a mechanical rasping action against tissue.

39. A surgical device comprising:
an active electrode; and
an insulating region adjacent the active electrode, the insulating region having a roughened surface for providing a mechanical rasping action against tissue.

40. A surgical device comprising an active electrode wherein:
the active electrode defines an envelope in a given plane, the given plane going through the active electrode, and
an electrical characteristic of the surgical device achieves a maximum for the given plane outside of the envelope.

41. The surgical device of claim 40 wherein the electrical characteristic is substantially uniform around a periphery of the active electrode when the electrical characteristic is measured in the given plane, the given plane being perpendicular to an engagement angle between the active electrode and a tissue surface.

42. The surgical device of claim 40 wherein the electrical characteristic measured at any point in the given plane that is at least 3/100 of an inch outside of the envelope drops off to no more than 60% of a maximum value for the electrical characteristic in the given plane.

43. A surgical device comprising:
a hand wand;
a shaft rotatably coupled to the hand wand and continuously rotatable with respect to the hand wand, wherein the shaft defines an aspiration lumen and the shaft is adapted to be inserted into a joint in a body;
a rotation control coupled to the shaft for rotating the shaft;
a tube coupled to the shaft, the tube defining a lumen in communication with the aspiration lumen;
a suction control coupled to the tube for controlling suction through the aspiration lumen;
an active electrode coupled to the shaft; and
a power control coupled to the hand wand for controlling power applied to the active electrode.

44. The surgical device of claim 43 wherein:
the rotation control comprises a knob,
the suction control comprises a valve, and
the power control comprises a push button.

45. A system comprising an adapter configured to be electrically coupled to an active electrode and to a generator, wherein the adapter includes circuitry to convert monopolar output from the generator into bipolar output for the active electrode.

46. The system of claim 45 wherein the circuitry is adapted to convert substantially constant power output from the generator into substantially constant voltage output.

47. The system of claim 45 wherein the adapter is configured to be electrically coupled to a return electrode.

48. The system of claim 47 further comprising the active electrode and the return electrode, the active electrode and the return electrode both being electrically coupled to the adapter.
